(19) United States
(12) Patent Application Publication
(10) Pub. No.: US 2025/0000708 A1
(43) Pub. Date: Jan. 2, 2025

(54) ADJUSTABLE COMPRESSION BAND, APPARATUS AND GARMENT

(71) Applicant: Human Aerospace Pty Ltd, Camberwell (AU)

(72) Inventors: Braid MACRAE, Camberwell (AU); James WALDIE, Camberwell (AU)

(21) Appl. No.: 18/759,159

(22) Filed: Jun. 28, 2024

(30) Foreign Application Priority Data
Jun. 29, 2023 (AU) 2023902065

(51) Int. Cl. A61F 13/01; A61F 13/00

(52) U.S. Cl. CPC A61F 13/01038; A61F 13/00051

(57) ABSTRACT

Embodiments relate to an adjustable compression band comprising: a band of flexible material configured to extend circumferentially around a body part of a user, at least part of the band being resiliently stretchable in the circumferential direction; a baseline circumference indicator for determining a baseline circumference of the body part; and an adjustment mechanism for adjusting a tension of the band in the circumferential direction to apply pressure to the body part. The adjustment mechanism comprises an inextensible element, which is relatively inextensible in comparison with the resiliently stretchable part of the band, and which comprises a tension indicator for indicating a target tension adjustment setting corresponding to the determined baseline circumference, the target tension adjustment setting corresponding to a tension required for the band to apply a predetermined interface pressure to the body part based on the determined baseline circumference.

### TECHNICAL FIELD

Embodiments generally relate to adjustable compression bands, garments comprising such compression bands, and methods of adjusting a compression band to apply a predetermined pressure to a body part of a user.

### BACKGROUND

Compression garments have a range of uses including for therapeutic, prophylactic, or performance purposes. These garments apply pressure to the body at the body-garment interface and, thereby, compress the underlying body tissue(s). They can sometimes be tailored to suit individuals, but typically are not adjustable, or where they are adjustable, require additional equipment to accurately measure and adjust the pressure applied to the body.

There are other devices for applying a specific interface pressure to a body part of a user, but they are relatively complex, expensive, bulky and heavy, so are not well-suited to applications such as human spaceflight, where tissue compression is required, and can be disadvantageous in other applications as well.

It is desired to address or ameliorate one or more shortcomings or disadvantages associated with existing compression bands, garments and devices, or to at least provide a useful alternative.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Some embodiments relate to an adjustable compression band comprising:
a band of flexible material configured to extend circumferentially around a body part of a user; and
an adjustment mechanism for adjusting a tension of the band in the circumferential direction to apply pressure to the body part,
wherein at least part of the band is resiliently stretchable in the circumferential direction, and
wherein the band comprises a baseline circumference indicator for determining a baseline circumference of the body part, and a tension indicator for indicating a target tension adjustment setting corresponding to the determined baseline circumference, the target tension adjustment setting corresponding to a tension required for the band to apply a predetermined interface pressure to the body part based on the determined baseline circumference.

In some embodiments, the adjustment mechanism comprises the baseline circumference indicator. In some embodiments, the band comprises the baseline circumference indicator.

In some embodiments, the adjustment mechanism comprises the tension indicator. In some embodiments, the band comprises the tension indicator.

Some embodiments relate to an adjustable compression band comprising:
a band of flexible material configured to extend circumferentially around a body part of a user, at least part of the band being resiliently stretchable in the circumferential direction;
a baseline circumference indicator for determining a baseline circumference of the body part; and
an adjustment mechanism for adjusting a tension of the band in the circumferential direction to apply pressure to the body part,
wherein the adjustment mechanism comprises an inextensible element, which is relatively inextensible in comparison with the resiliently stretchable part of the band, and which comprises a tension indicator for indicating a target tension adjustment setting corresponding to the determined baseline circumference, the target tension adjustment setting corresponding to a tension required for the band to apply a predetermined interface pressure to the body part based on the determined baseline circumference.

In some embodiments, the inextensible element comprises the baseline circumference indicator.

In some embodiments, the adjustment mechanism comprises a ratcheting tensioner and the inextensible element comprises a tensioning dial of the tensioner with the tension indicator comprising markings on the dial.

In some embodiments, the inextensible element comprises a relatively inextensible part of the band with the tension indicator comprising markings on the relatively inextensible part of the band.

In some embodiments, the inextensible element comprises a relatively inextensible cord coupling ends of the band together with the tension indicator comprising markings on the inextensible cord. For example, laced up eyelets.

In some embodiments, at least part of the band (or other element) is relatively inextensible (or inelastic) in the circumferential direction in comparison with the resiliently stretchable part of the band. Throughout the specification, the terms inextensible, inelastic, or non-stretch refer to materials or portions of the band which are substantially inextensible/inelastic, or relatively inextensible/inelastic in comparison with other portions of the band. The inextensible portion is not required to be strictly inextensible or inelastic and, in practice, may be slightly extensible or stretchable, and may even be slightly elastic or resiliently stretchable, but much less so than the resiliently stretchable part of the band.

For example, the relatively inextensible portion of the band may be configured to experience a circumferential strain of less than 5%, less than 3%, less than 2%, or less than 1%. The relatively elastic (or resiliently stretchable) portion may be configured to be put under relatively higher strains for the range of tensions applied to the band in use.

For a certain tension of the band in the circumferential direction, the ratio of the circumferential strain of the relatively inextensible portion to the circumferential strain of the resiliently stretchable portion may be less than 0.5, less than 0.3, less than 0.2, less than 0.1, less than 0.05, or less than 0.01, for example.

The resiliently stretchable part of the band may also be referred to as the elastic or relatively elastic part, or the extensible part of the band. Throughout the specification, the term elastic is referring to a physical property of the band, i.e., a combination of the materials used and their form (e.g., fabric/membrane, thickness, number of layers, etc.) resulting in the physical property of elasticity, being the relative extent of strain in a certain direction (circumferential or lateral) for a given tension applied in that direction, while being able to resiliently returning to the original dimensions (or close to the original dimensions) when the tension is removed.

In some embodiments, the inextensible part of the band comprises the baseline circumference indicator and/or target tension indicator.

In some embodiments the compression band, further comprises a baseline tension indicator configured to indicate when a predetermine threshold baseline tension is applied to the band, the baseline tension being a suitable tension for using the baseline circumference indicator for determining the baseline circumference of the body part.

In some embodiments, the baseline tension indicator comprises a first sheet of resiliently stretchable material coupled in parallel to a second sheet of relatively inextensible material such that the first sheet is shorter than the second sheet in the circumferential direction of the band, when not under tension, and
wherein the first and second sheets are arranged in series with the rest of the band and configured such that, when the baseline tension is applied to the baseline tension indicator and the band for determining the baseline circumference using the baseline circumference indicator, the first sheet is extended to the same circumferential length as the second sheet.

In some embodiments, a length of the resiliently stretchable part of the band in the circumferential direction varies across a lateral width of the band such that the predetermined interface pressure applied to the body part varies across the width of the band.

In some embodiments, a circumference of the band varies across a lateral width of the band.

In some embodiments, the varying circumference of the band across the lateral width of the band is configured such that the predetermined interface pressure applied to the body part varies across the width of the band.

In some embodiments, the predetermined interface pressure associated with the target tension is a first predetermined pressure associated with a first target tension and corresponding target tension adjustment settings associated with corresponding baseline circumferences, and the tension indicator further comprises a second target tension with corresponding target tension adjustment settings associated with corresponding baseline circumferences for the band to apply a second predetermined interface pressure to the body part.

In some embodiments, the baseline circumference indicator comprises a set of baseline circumference markings, and the tension indicator comprises a set of target tension markings, each of the target tension markings indicating the target tension adjustment setting associated with the baseline circumference of a corresponding one of the baseline circumference markings to apply the corresponding predetermined interface pressure.

In some embodiments, the target tension markings comprise a first set of markings corresponding to the first predetermined pressure, and a second set of markings corresponding to the second predetermined pressure.

Some embodiments relate to an adjustable compression band comprising:
a band of flexible material configured to extend circumferentially around a body part of a user; and
an adjustment mechanism for adjusting a tension of the band in the circumferential direction to apply pressure to the body part,
wherein at least part of the band is resiliently stretchable in the circumferential direction, and
wherein the band comprises indicator markings including a first set of markings, for measuring a baseline circumference of the body part, and a second set of markings indicating a target tension adjustment setting for the band to apply a predetermined interface pressure to the body part, each of the second set of markings indicating the target tension adjustment setting associated with the baseline circumference of a corresponding one of the first set of markings to apply the predetermined interface pressure.

In some embodiments, the inextensible part of the band comprises the indicator markings. In some embodiments, the extensible (or resiliently stretchable) part of the band comprises the indicator markings.

In some embodiments, a length of the inextensible part of the band in the circumferential direction varies across a lateral width of the band such that the predetermined interface pressure applied to the body part varies across the width of the band. In some embodiments, a length of the inextensible part of the band in the circumferential direction varies across a lateral width of the band such that the predetermined interface pressure applied to the body part is uniform across the width of the band. In some embodiments, the circumferential length of the inextensible part is constant across the lateral width of the band.

In some embodiments, a length of the extensible part of the band in the circumferential direction varies across a lateral width of the band such that the predetermined interface pressure applied to the body part varies across the width of the band. In some embodiments, a length of the extensible part of the band in the circumferential direction varies across a lateral width of the band such that the predetermined interface pressure applied to the body part is uniform across the width of the band. In some embodiments, the circumferential length of the extensible part is constant across the lateral width of the band.

In some embodiments, a circumference of the band varies across a lateral width of the band. In some embodiments, the circumference of the band is constant across a lateral width of the band.

In some embodiments, the band may define a varying circumference with constant circumferential length of the extensible portion and varying circumferential length of the inextensible portion.

In some embodiments, the band may define a varying circumference with varying circumferential length of the extensible portion and constant circumferential length of the inextensible portion.

In some embodiments, the band may define a varying circumference with varying circumferential length of both the extensible and inextensible portions.

In some embodiments, the band may define a constant circumference with varying circumferential length of the extensible and inextensible portions. For example, this configuration may be used to apply a varying pressure profile to a body part of substantially constant circumference.

In some embodiments, the band may define a constant circumference with constant circumferential length of each of the extensible and inextensible portions. For example, this configuration may be used to apply a uniform pressure profile to a body part of substantially constant circumference, or to apply a varying pressure profile to a body part of varying circumference.

In some embodiments, the band may have a constant lateral width around the circumference of the band. In some embodiments, side edges of the band may be straight. For example, the side edges may be parallel, i.e., corresponding to a constant lateral width of band.

In some embodiments, the band may have a varying lateral width around the circumference of the band. In some embodiments, the side edges 116 may be curved, i.e., corresponding to a varying lateral width of band 110. For example, the side edges 116 may curve inwards or outwards, or a combination of both inward and outward curves. In some embodiments, one side edge may be straight and the other side edge may be curved.

In some embodiments, the varying circumference of the band across the lateral width of the band is configured such that the predetermined interface pressure applied to the body part varies across the width of the band. In some embodiments, the varying circumference of the band across the lateral width of the band is configured such that the predetermined interface pressure applied to the body part is uniform across the width of the band.

In some embodiments, the predetermined interface pressure associated with the second set of markings is a first predetermined pressure, and
wherein the indicator markings further comprise a third set of markings indicating a target tension adjustment setting for the band to apply a second predetermined interface pressure to the body part, each of the third set of markings indicating a target tension adjustment setting associated with the baseline circumference of a corresponding one of the first set of markings to apply the second predetermined interface pressure.

The indicator markings may further comprise a fourth, fifth, sixth, seventh set of markings associated with further predetermined interface pressures (or pressure profiles). The indicator markings may comprise any suitable number of sets of markings corresponding to predetermined interface pressures (or pressure profiles).

In some embodiments, each set of markings includes a plurality of markings covering a range of baseline circumferences to allow for expected dimensional changes of the body part, or to allow for dimensional variation between users.

In some embodiments, each of the markings in each set of markings includes a visual similarity with the or each corresponding marking in the or each other set of markings. For example, corresponding colours, patterns, symbols, numbers, letters, shapes, etc.

In some embodiments, the adjustment mechanism is configured to adjust the distance in the circumferential direction between two parts of the band. This adjustment of the circumferential distance between two parts of the band applies a strain to the extensible portion to achieve a corresponding tension in the band and a corresponding interface pressure applied to the body part.

In some embodiments, the adjustment mechanism comprises fasteners disposed at a range of circumferential locations on the band allowing one part of the band to be fastened to another part of the band to select and set the tension of the band. For example, by adjusting the circumferential distance between the two parts of the band. The fasteners may comprise any suitable fasteners such as, hook-and-loop fasteners (e.g., Velcro); hooks and eyelets; press studs; snap fasteners; or cohesive fabric, for example.

In some embodiments, the adjustment mechanism comprises a buckle. The buckle may comprise a loop buckle to allow the band to be folded back on itself under tension. The buckle may comprise a locking buckle to set the tension at a selected adjustment setting.

In some embodiments, the adjustment mechanism comprises eyelets laced together to adjust the tension of the band. For example, the eyelets may be laced together with string or cord, similar to shoe laces.

In some embodiments, the band is configured to surround and apply pressure to part of a limb of the user. For example, an arm or leg or part thereof. In some embodiments, the band is configured to surround and apply pressure to part of the user's torso.

Some embodiments relate to a garment comprising one or more adjustable compression bands according to any one of the described embodiments. The garment may comprise a sleeve, arm sleeve, leg sleeve, abdominal sleeve, trousers, jacket, suit, compression suit, full body suit, partial body suit, sleeveless body suit, orthostatic intolerance garment, or extravehicular activity suit (EVA suit), for example.

The garment may comprise non-adjustable portions connected to the one or more compression bands. The non-adjustable portions may be disposed between the compression bands. In some embodiments, the adjustable compression bands may be integrally formed with the non-adjustable portions in the garment.

In some embodiments, the garment comprises a plurality the adjustable compression bands arranged in parallel and integrally formed with each other.

Some embodiments relate to a method of applying an interface pressure to a body part of a user, the method comprising:
placing a compression band according to any one of the described embodiments around the body part of the user;
adjusting the circumference of the band to a neutral configuration to determine a baseline circumference of the body part indicated by the baseline circumference indicator; and
adjusting the tension of the band to a corresponding target tension indicated by the target tension indicator to apply the predetermined pressure to the body part.

Some embodiments relate to a method of applying a predetermined interface pressure to a body part of a user, the method comprising:
placing a compression band according to any one of the described embodiments around the body part of the user;
adjusting the circumference of the band to a neutral configuration to determine a baseline circumference of the body part indicated by the first set of indicator markings; and
adjusting the tension of the band to a target tension indicated by one of the second set of indicator markings corresponding to the baseline circumference.

Adjusting the circumference of the band to the neutral configuration may comprise taking up any slack in the band so that the circumference of the band matches the circumference of the body part without applying significant pressure to the body part and without applying significant strain to the band. That is, negligible strain in the band and negligible pressure applied to the body part.

### BRIEF DESCRIPTION OF DRAWINGS

Some embodiments will now be described, for illustrative purposes only, with reference to the drawings, in which:

FIG. 1 shows a general schematic diagram of an adjustable compression band, according to some embodiments;

FIG. 2A shows a front side of an adjustable compression band (outward facing in use), according to some embodiments;

FIG. 2B shows a back side of the adjustable compression band of FIG. 2A (inward facing in use);

FIG. 2C shows the adjustable compression band of FIG. 2A on a body part of a user illustrating the baseline circumference of the body part;

FIG. 2D shows the adjustable compression band of FIG. 2A on the body part set to a tension adjustment setting corresponding to the baseline circumference to apply a predetermined interface pressure (or pressure profile) to the body part;

FIG. 2E shows the adjustable compression band of FIG. 2A in use applying compression to part of a lower leg of a user;

FIG. 3A shows an adjustable compression band according to some embodiments;

FIG. 3B shows the adjustable compression band of FIG. 3A on a body part of a user illustrating the baseline circumference of the body part;

FIG. 3C shows the adjustable compression band of FIG. 3A on the body part set to a tension adjustment setting corresponding to the baseline circumference to apply a predetermined interface pressure (or pressure profile) to the body part;

FIG. 4A shows an adjustable compression band according to some embodiments;

FIG. 4B shows the adjustable compression band of FIG. 4A on a body part of a user illustrating the baseline circumference of the body part;

FIG. 4C shows the adjustable compression band of FIG. 4A on the body part set to a tension adjustment setting corresponding to the baseline circumference to apply a predetermined interface pressure (or pressure profile) to the body part;

FIG. 4D shows the adjustable compression band of FIG. 4A in an unstrained configuration with illustrative dimensions for calculating the location of indicator markings;

FIG. 4E shows the adjustable compression band of FIG. 4A in a strained configuration with illustrative dimensions for calculating the location of indicator markings and showing the change in circumferential length of the band;

FIGS. 5A to 5D show different embodiments of the compression band with different shapes to accommodate differently shaped body parts;

FIGS. 6A to 6C show illustrative dimensions for calculating the location of indicator markings for an adjustable compression band which varies in circumference across a lateral width of the band, with FIG. 6B showing the band in an unstrained configuration and FIG. 6C showing the band in a strained configuration;

FIG. 7A shows a compression sleeve comprising a plurality of compression bands, according to some embodiments;

FIG. 7B shows the compression sleeve of FIG. 7A on a lower leg of a user;

FIG. 8 shows a variety of different garments comprising one or more compression bands, according to some embodiments;

FIG. 9 shows a full body compression suit comprising a plurality of adjustable compression bands, according to some embodiments;

FIG. 10 shows a flat plan view of a compression band, according to some embodiments;

FIG. 11A shows a laced compression band with indicator markings on the lace in a configuration for determining a baseline circumference of a body part;

FIG. 11B shows the laced compression band of FIG. 11A in a configuration for setting a target tension to apply a predetermined interface pressure to the body part;

FIG. 12A shows a perspective schematic illustration of a baseline tension indicator when not under tension;

FIG. 12B shows the baseline tension indicator of FIG. 12A with the baseline tension applied;

FIG. 13 shows the baseline tension indicator of FIG. 12A incorporated into a compression band, according to some embodiments;

FIG. 14 shows a removable baseline tension indicator coupled to a compression band for determining the baseline circumference, according to some embodiments;

FIG. 15 shows a baseline tension indicator incorporated into a compression band, according to some embodiments;

FIG. 16 shows a baseline tension indicator incorporated into a compression band, according to some embodiments; and

FIG. 17 shows a baseline tension indicator incorporated into a compression band, according to some embodiments.

### DESCRIPTION OF EMBODIMENTS

This disclosure defines a general principle for readily adjusting the interface pressure applied by a compression band (or garment) to the body part of the user. The tension can be adjusted to apply a predetermined interface pressure accounting for the baseline circumference of the body part based on tension indicators corresponding to different baseline circumferences and adjustment settings.

This allows for a predetermined interface pressure to be applied to the body part of a user without the need for separate measurement equipment. It also allows the compression band (or garment) to be adjusted as the circumference of the body part changes over time, which may occur due to muscle atrophy or oedema, for example, or to accommodate dimensional variation between different users.

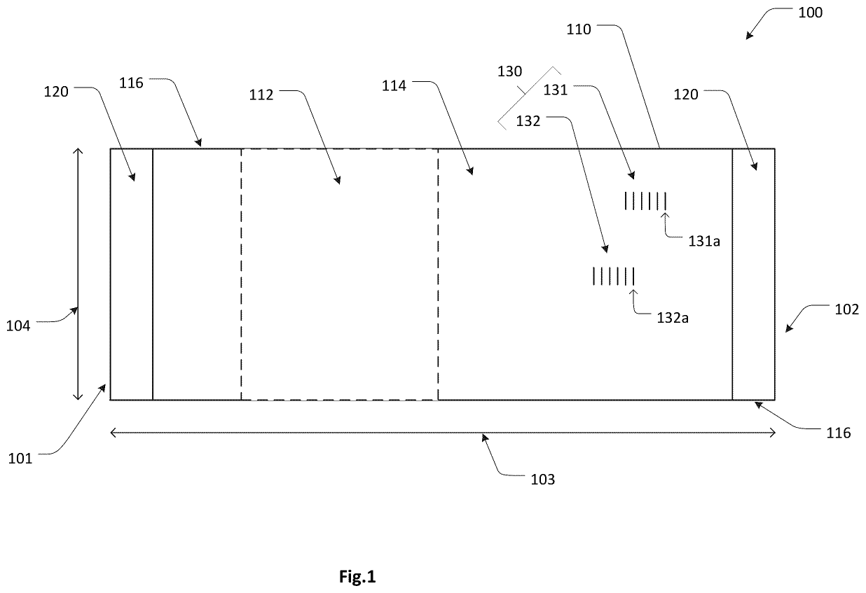

This concept may be implemented in many different arrangements and configurations of compression bands with various materials, adjustment mechanisms, and tension indicators such as indicator markings. A generalised example with indicator markings is shown in FIG. 1 with other embodiments of the compression band shown in the following figures. However, it should be understood that many other combinations of features could be included in an adjustable compression band incorporating the adjustment principle of this disclosure.

Some embodiments relate to a method of applying an interface pressure to a body part of a user, the method comprising:
placing a compression band around the body part of the user, the compression band comprising a baseline circumference indicator, a tension adjustment mechanism, and a target tension indicator configured to indicate a target tension associated with a particular baseline circumference and corresponding to a predetermined interface pressure to be applied to the body part;
adjusting the circumference of the band to a neutral configuration to determine a baseline circumference of the body part indicated by the baseline circumference indicator; and
adjusting the tension of the band to a corresponding target tension indicated by the target tension indicator.

The baseline circumference may also be referred to as the current circumference of the body part. That is, the circumference of the body part at the time of donning the compression band. The compression band may be configured to accommodate a range of different baseline circumferences to allow for different sized body parts, variation between users, or changes in a particular user's body part over time, such as dimensional variations due to muscle atrophy, oedema, muscle growth, or other changes in tissues or fluids in the tissues.

The baseline circumference indicator may comprise any suitable means for measuring the circumference of the body part, and may simply comprise a scale or part of a scale with markings indicating various baseline circumferences. The markings may include units, but not necessarily so, and may simply include markings at a range of arbitrary circumferences covering a suitable range for a given application. The marked baseline circumferences may be associated with specific target tensions, as described in further detail and various embodiments throughout the specification.

The baseline circumference may be determined at a neutral configuration. That is, in a natural, uncompressed state of the body part of the user. The baseline circumference may be determined by placing or wrapping the compression band around the body part to form a snug fit and taking up any slack in the compression band. In practice, this may result in a small interface pressure being applied to the body part and/or a small strain put on the compression band. However, nominally this can be assumed to be zero, or negligibly small for the purposes of applying a corresponding target tension to apply a predetermined interface pressure to the body part.

The predetermined pressure may be a specific interface pressure or a pressure profile (which may vary across a lateral width of the compression band) desired to be applied to the body part for therapeutic or other reasons, as discussed elsewhere in the specification. The interface pressure may be applied to the body part to compress muscle and/or other tissues in the body part. The target tension required to apply the predetermined pressure or pressure profile may be calculated based on the baseline circumference. Similarly, there may be a strain in the band associated with the target tension, which may also be calculated based on the baseline circumference. Sample calculations are set out in the specification, for example.

In this way, a set of target tensions (and optionally associated strains) can be calculated ahead of time for each of a corresponding set of baseline circumferences in order to apply a predetermined pressure or pressure profile to the body part.

When a user utilises the compression band to apply the predetermined pressure or pressure profile to their body part, they can simply apply the method using the compression band to determine the baseline circumference of the body part at that time, and then adjust the tension in the band to reach the target tension corresponding to the determined baseline circumference, as indicated by the target tension indicator.

The adjustment mechanism may comprise any suitable mechanism for adjusting tension in the band (and the corresponding pressure applied to the body part), and several examples are described in the specification. The adjustment mechanism may be configured to adjust the tension in the band by adjusting the circumferential distance between two parts of the band, i.e., bringing the two parts closer together or allowing them to move further apart. Various embodiments of this type are disclosed herein.

The tension indicator may comprise any suitable means for indicating the tension in the compression band and how it corresponds to the target tension associated with the baseline circumference. Unless otherwise indicated, references to the tension in the compression band or target tension are intended to refer to the circumferential tension, or the tension in the circumferential direction (extending around the body part in use).

The embodiments described herein focus on target tension indicator markings on the band itself, which may be considered to form part of the adjustment mechanism. In other embodiments, the tension indicator could comprise tension indicator markings on a different part of the adjustment mechanism rather than the band. In other embodiments, the tension indicator may comprise one or more strain gauges or stress gauges disposed in or on the band to indicate the tension in the band.

For each predetermined pressure or pressure profile and baseline circumference, there is an associated target tension with clearly defined relationships between these parameters allowing the target tension to be calculated ahead of time and indicated with the target tension indicator so that the calculations are not required at the time of donning the compression band to apply the predetermined pressure or pressure profile.

In the compression bands described below, there is also a strain associated with each target tension, the magnitude of which depends on the material properties and dimensions of the band. The sample calculations presented below illustrate the relationships between strain and tension in the band for various baseline circumferences and the corresponding predetermined pressure to be applied. The target tensions associated with the baseline circumferences are displayed on the tension indicator facilitating adjustment of the compression band tension to apply the predetermined pressure.

In some embodiments, the strain in the band may be used to set the target tension. For example, the compression band may comprise one or more strain gauges configured to measure the strain in part of the band, and indicate a particular target strain associated with a corresponding baseline circumference and the corresponding target tension required to apply the predetermined interface pressure.

In some embodiments, the corresponding strain may be determined using indicator markings, as discussed below.

In some embodiments, the tension in the band may be measured via a tension meter allowing the tension in the band to be adjusted and set to the target tension required to apply the predetermined interface pressure.

In some embodiments, the stress in the band may be measured via a stress gauge allowing the tension in the band to be adjusted and set to the target tension required to apply the predetermined interface pressure.

The interface pressure, tension, stress and strain are all related to each other, with one approach to modelling these relationships set out in the calculations below. The adjustment mechanism is configured to allow the tension in the band to be adjusted (and optionally also set) to a target tension. The tension in the band results in an interface pressure being applied to the body part, and the target tension is associated with a predetermined interface pressure or pressure profile. Depending on the materials and form of the band, the tension in the band may also result in strain in the band. As mentioned above, the strain may be used to determine the tension in the band, and there may be a target strain associated with a corresponding target tension and predetermined pressure applied to the body part.

It will be appreciated that there are many varied combinations of different compression band configurations, adjustment mechanisms, and tension indicators that could be employed to implement this principle for applying and adjusting an interface pressure to a body part. Some examples are presented in the following embodiments.

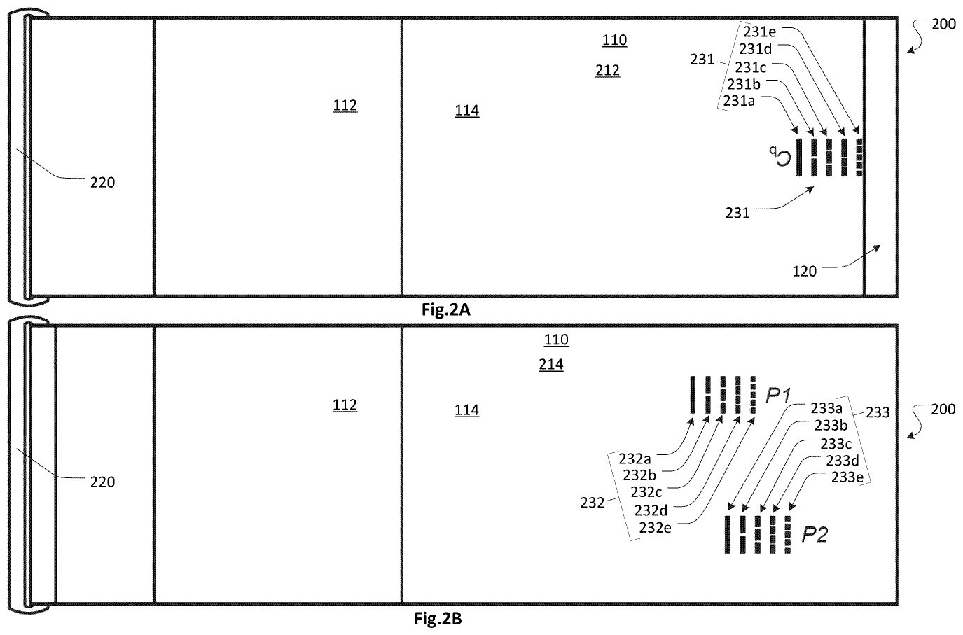

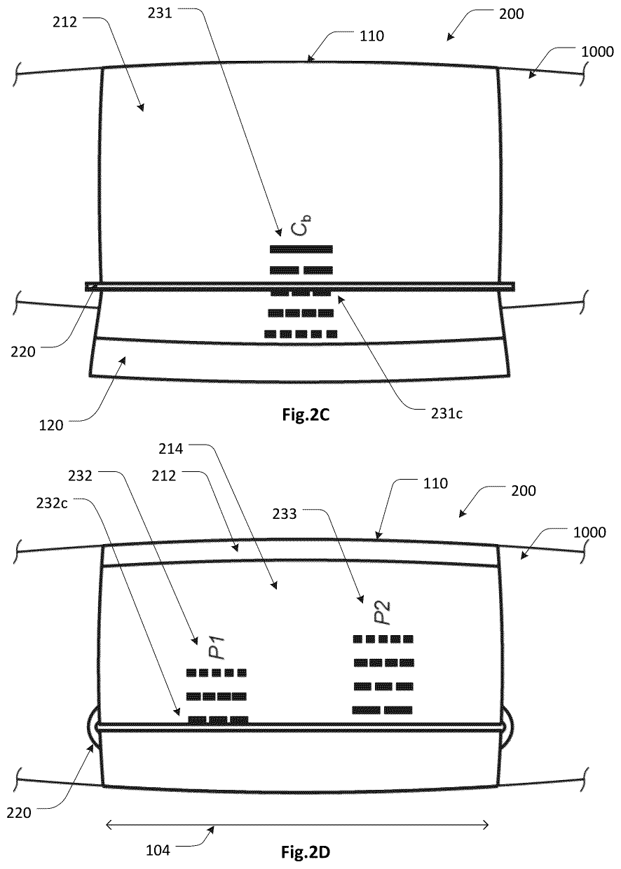

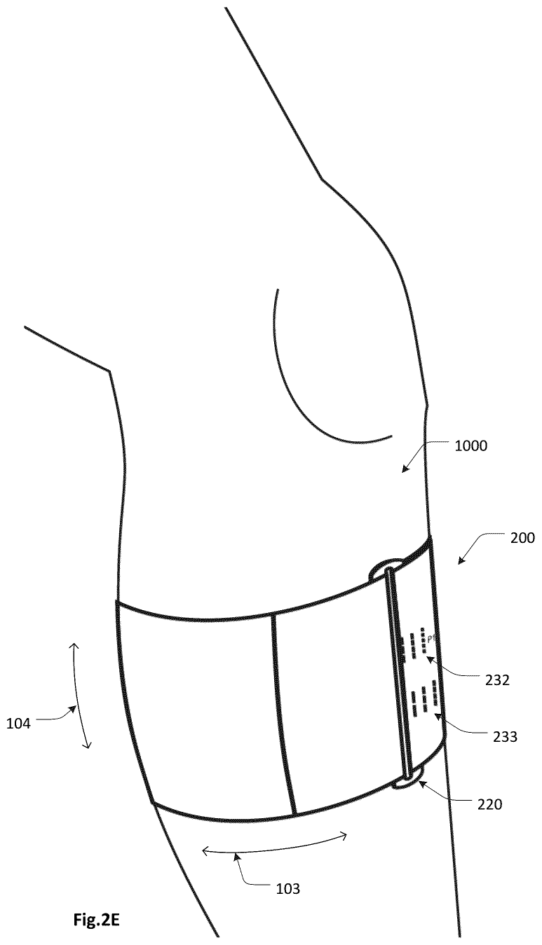

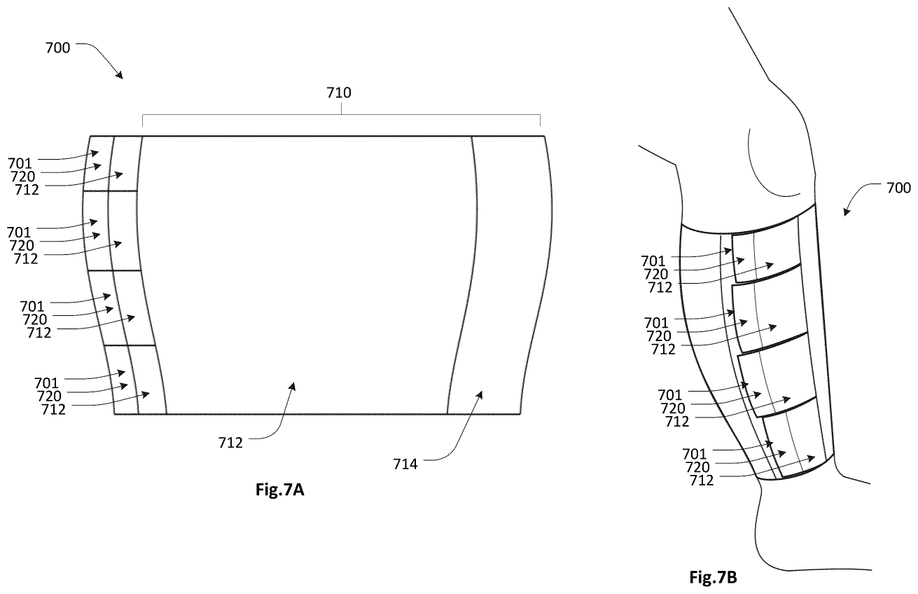

Referring to FIG. 1, an adjustable compression band 100 is shown according to some embodiments. The compression band 100 may be individually placed on (or applied to) a body part of a user as shown in the examples in FIGS. 2C, 2D, 2E, 3B, 3C, 4B, 4C. In other embodiments, the compression band 100 may form part of a garment, such as those shown in FIGS. 7A-7B, 8 and 9, as described further below.

The compression band 100 is illustrated and described as a generalised version with features similar to several different embodiments, but does not illustrate some of the other embodiments described above. For example, those with baseline circumference indicators or tension indicators other than markings, or those with the baseline circumference indicators and/or tension indicators formed on a different part of the adjustment mechanism other than the band, or those with different adjustment mechanisms.

The compression band 100 illustrated in FIG. 1 shows a simple version with markings on the band and a simple adjustment mechanism with fasteners.

The compression band 100 comprises a band 110 of flexible material configured to extend circumferentially around a body part of a user. For example, a limb or torso of the user.

The band 100 is shown laid out flat in FIG. 1 extending between a first end 101 and a second end 102. The circumferential direction 103 is shown by arrow 103, that is, the direction in which the band extends around body part of the user. The extent of the band 110 or parts thereof in the circumferential direction 103 may be referred to as the circumference, distance in the circumferential direction, circumferential length, or, in the calculations herein, it is referred to as the length. The lateral direction 104 (i.e., lateral to the circumferential direction 103) is shown by arrow 104. The lateral extent of the band 110 in the lateral direction 104 may be referred to as the width, lateral width, or, in the calculations herein, it is referred to as the height.

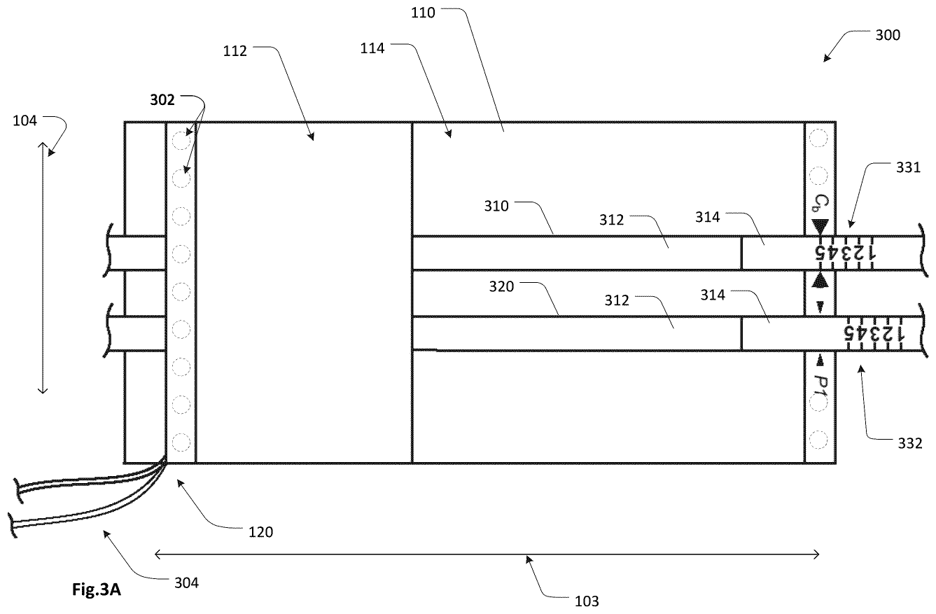

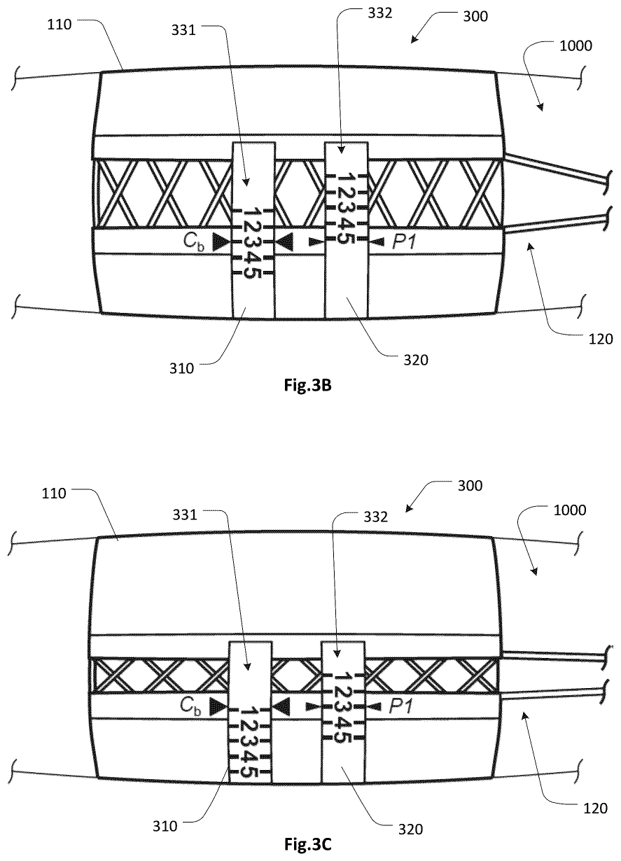

In some embodiments, the band 110 may comprise a continuous loop (i.e., with the first end 101 connected to the second end 102) which can be slipped onto the body part of the user and then adjusted. For example, compression band 300 shown in FIGS. 3A to 3C.

The compression band 100 comprises an adjustment mechanism 120 for adjusting a tension of the band 110 in the circumferential direction 103 to apply pressure to the body part. Several different suitable adjustment mechanisms are described below in relation to various embodiments, and any other suitable adjustment mechanism for adjusting the tension of the band 110 in the circumferential direction 103 may be used.

The adjustment mechanism 120 may also be configured to select and set the tension in the band 110, or fix the tension of the band 110 at a particular tension adjustment setting. In other embodiments, a separate fastener, fixing mechanism or locking mechanism may be employed to set the tension of the band 110 at a particular tension adjustment setting. That is, separate to the adjustment mechanism 120.

In FIG. 1, the adjustment mechanism 120 is illustrated as portions of the band 110 at each end 101, 102 of the band 110, which may comprise complimentary parts of a hook-and-loop fastener, such as Velcro, for example. The band 110 may be stretched to the desired strain (based on markings discussed below) and then fastened at a set strain (corresponding to a target tension) with the Velcro. Any other suitable adjustment mechanism 120 may be used for adjusting the tension in the band 110, including those discussed below in relation to other embodiments.

At least part of the band 110 is resiliently stretchable in the circumferential direction 103. In some embodiments, the entire circumferential length of the band 110 may be resiliently stretchable. In some embodiments, part of the band 110 may be relatively inextensible.

For example, the band 110 may comprise a relatively inextensible or inelastic portion 114 and a relatively extensible or elastic portion 112. Various materials and configurations are described in further detail below. In embodiments where the relatively inextensible portion 114 comprises the tension indicator markings, the relatively inextensible portion 114 may be considered to form part of the adjustment mechanism.

Unless otherwise specified, references to elastic or inelastic are intended to refer to the amount of strain in the circumferential direction 103 for a given circumferential tension. For example, under the expected range of band tension (or stress) in use of the compression band 110 (which may be up to 8000 N/m normalised across the lateral width, for example). The relatively inelastic portion may be configured to experience a circumferential strain of less than 5%, less than 3%, less than 2%, or less than 1%.

For a certain tension of the band 110 in the circumferential direction 103, the ratio of the circumferential strain of the relatively inelastic (or inextensible) portion to the circumferential strain of the relatively elastic (or extensible) portion may be less than 0.5, less than 0.3, less than 0.2, less than 0.1, less than 0.05, or less than 0.01, for example.

In some embodiments, the relatively elastic portion 112 may be relatively inelastic or inextensible in the lateral direction 104. In some embodiments, the relatively elastic portion 112 may be relatively elastic in the lateral direction 104.

In some embodiments, the relatively inextensible portion 114 may be relatively inelastic or inextensible in the lateral direction 104. In some embodiments, the relatively inextensible portion 114 may be relatively elastic (or resiliently stretchable/extensible) in the lateral direction 104.

Using material in the band 110 which is relatively elastic in the lateral direction 104 (in the elastic and/or inelastic portions 112, 114) may facilitate different parts of the band 110 across the lateral width of the band 110 stretching to different extents in the circumferential direction to accommodate variation in the circumference of the body part in the lateral direction 104 (i.e., along the length of the body part, limb or torso of the user) and also allow stretching in the lateral direction to accommodate flexing of body parts, such as joints, or variation in length along the body part, such as curved body parts, for example.

The compression band 100 is illustrated as rectangular in FIG. 1, corresponding to a constant circumference across the lateral width of the band 110 which would be generally cylindrical in use on a body part of a user. In some embodiments, the band 110 may define a constant circumference with constant circumferential length of the extensible and inextensible portions.

In various other embodiments, the compression band 100 may vary in circumference across the lateral width and/or vary in lateral width along the circumference in order to accommodate the variation in circumference and/or curvature of the body part of the user. For example, to accommodate a lower part of a leg with a relatively straight shin and a curved calf which tapers towards the ankle.

The varying circumference may be achieved with the extensible portion 112 and/or the inextensible portion 114 varying in circumferential length across the lateral width of the band 110.

In some embodiments, the band 110 may define a varying circumference with constant circumferential length of the extensible portion and varying circumferential length of the inextensible portion.

In some embodiments, the band 110 may define a varying circumference with varying circumferential length of the extensible portion and constant circumferential length of the inextensible portion.

In some embodiments, the band 110 may define a varying circumference with varying circumferential length of both the extensible and inextensible portions.

In some embodiments, the band 110 may define a constant circumference with varying circumferential length of the extensible and inextensible portions. For example, this configuration may be used to apply a varying pressure profile (discussed further below) to a body part of substantially constant circumference.

In some embodiments, side edges 116 of the band 110 may be straight. For example, the side edges 116 may be parallel, i.e., corresponding to a constant lateral width of band 110.

In some embodiments, the side edges 116 may be curved, i.e., corresponding to a varying lateral width of band 110. For example, the side edges 116 may curve inwards or outwards, or a combination of both inward and outward curves. In some embodiments, one side edge may be straight while the other side edge is curved.

The varying lateral width may be achieved with the extensible portion 112 and/or the inextensible portion 114 varying in lateral width across the circumferential length of the band 110.

The band 110 comprises indicator markings 130 including a first set of markings 131, for measuring a baseline circumference of the body part, and a second set of markings 132 indicating a target tension adjustment setting for the band 110 to apply a predetermined interface pressure to the body part.

Each of the second set of markings 132 (e.g. 132a) indicates the target tension adjustment setting associated with the baseline circumference of a corresponding one of the first set of markings 131 (e.g. 131a) to apply the predetermined interface pressure.

The location of the second set of markings 132 may be calculated as described further below based on the strain (i.e., change in circumferential length of the band 110) corresponding to the desired tension for applying the predetermined interface pressure.

As discussed above, the band 110 may be configured to apply a constant interface pressure across the lateral width of the band 110, or in some embodiments, may be configured to apply a predetermined pressure profile to the body part with different interface pressures applied across the lateral width of the band 110.

The first set of markings 131 is used to determine the baseline circumference of the body part. The first set of markings 131 may include a plurality of markings to cover a range of baseline circumferences to suit different users, or to account for changes in circumference of the body part over time, such as due to atrophy or oedema, for example.

During the treatment of lymphodema on Earth, for example, the circumference of a user's body part(s) (e.g., limbs) may decrease with the reduction of local swelling over the duration of a compression treatment. The range of baseline circumferences may be configured to cover the expected changes so that the compression band can be readjusted based on the current baseline circumference at various intervals in the treatment.

In human spaceflight, for example, the circumference of a user's body part(s) (e.g., limbs) may change over time in low gravity or zero gravity due to muscle atrophy or hypertrophy, fluid shifting, fluid losses or fluid retention. For example, the circumference of lower limbs (legs) may decrease while the circumference of upper limbs (arms) may increase.

The first set of markings 131 may cover the range of expected baseline circumferences of a body part over the duration of a mission, so that the compression band 110 can be adjusted by checking the baseline circumference at different stages and then setting the tension for the predetermined pressure corresponding to the baseline circumference at each stage.

The second set of markings 132 includes a plurality of target tension indicator markings, each corresponding to one of the baseline circumference markings 131 to set the tension for the band 110 to apply the predetermined interface pressure or pressure profile. The second set of markings 132 may include 1, 2, 3, 4, 5, 6, 7, 8, 9 or more markings each associated with a corresponding one of first set of markings 131, for example, the first set of markings 131 may include 131a, 131b, 131c, 131d, 131e, 131f, 131g, and the second set of marking 132 may include corresponding markings 132a, 132b, 132c, 132d, 132e, 132f, 132g.

The band 110 may include further sets of markings corresponding to different predetermined interface pressures or pressure profiles. The various sets of target tension indicator markings may correspond to different interface pressures or pressure profiles for different treatments, or to suit different pressure or gravity environments. For example, when the user is experiencing high acceleration or deceleration; different weights due to different gravity environments (lower or higher gravity) on different planets or moons, or low gravity, micro-gravity or zero-gravity in orbit or during other spaceflight; or different atmospheric pressures, such as in a space-craft, on a planet with different atmospheric pressure or in low pressure or vacuum pressure.

The band 110 may comprise 2, 3, 4, 5, 6, 7, 8, 9 or more sets of markings corresponding to different predetermined interface pressures or pressure profiles. For example, the band 110 may include a first set of markings for determining the baseline circumference; a second set of markings for applying an interface pressure for the high acceleration of launch; a third set of markings for applying an interface pressure for zero-gravity and cabin pressure in a spacecraft; a fourth set of markings for applying an interface pressure for zero-gravity in a vacuum environment; a fifth set of markings for applying an interface pressure for Moon gravity and vacuum; and a sixth set of markings for applying an interface pressure for Mars gravity and atmosphere, or any combination of these situations and corresponding markings.

In another example, the band 110 may include a first set of markings for determining the baseline circumference; a second set of markings for applying an interface pressure for Moon gravity and cabin ambient pressure; a third set of markings for applying an interface pressure for Mars gravity and cabin ambient pressure; and a fourth set of markings for applying an interface pressure for Earth gravity and atmosphere.

In another example, the band 110 may include a first set of markings for determining the baseline circumference; a second set of markings for applying an interface pressure for zero-gravity in a vacuum environment; a third set of markings for applying an interface pressure for Moon gravity and vacuum; and a fourth set of markings for applying an interface pressure for Mars gravity and atmosphere.

In another example, the band 110 may include a first set of markings for determining the baseline circumference; a second set of markings for applying an interface pressure during and following return to Earth from microgravity for a first period (e.g., the first 24 hours after return); a third set of markings for applying a second different (e.g., lower) interface pressure for a second period (e.g., 24-48 hours after return to Earth); and (optionally) a fourth set of markings for applying a third different (e.g., further lower) interface pressure for a third period (e.g., 48-72 hours after return to Earth).

The indicator markings 130 may be provided on the relatively inextensible portion 114 of the band 110, or on the extensible part of the band 110, e.g., in embodiments with an entirely elastic/extensible band 110. Calculations are set out below for indicator markings on the relatively inelastic/inextensible portion 114, with the assumption that the strain of the inextensible portion 114 is negligible compared with the strain of the extensible portion 112. These calculations could be modified to account for strain between markings if they are to be provided on an extensible portion.

Any suitable material may be used for the band 110, extensible portion 112 and inextensible portion 114. The band 110 may comprise a thin film or sheet of flexible material. The band 110 (extensible portion 112 and/or inextensible portion 114) may be formed of a membrane with suitable properties of flexibility and elasticity, such as polymers, elastomers, natural polymers, synthetic polymers, continuous polymer film or sheet, rubber, elastane, or latex, for example. Alternatively (or additionally) various fabrics may be used, including woven or knitted fabrics, with natural and/or synthetic fibres, yarns, or monofilaments. Some suitable materials for fabrics include cotton, linen, polyester, nylon, polyamide, elastane, rubber, spandex, and lycra, though any suitable materials may be used for the extensible and inextensible portions.

In some embodiments, the inextensible portion 114 may comprise relatively inelastic (and inextensible) fibres, yarns, or filaments oriented in the circumferential direction 103 and relatively elastic (and extensible) fibres, yarns, or filaments oriented in the lateral direction 104. This may allow for some sheer movement between adjacent circumferential fibres, yarns, or filaments allowing the extensible portion 112 to extend by different amounts in the circumferential direction, for example, to apply a varied pressure profile across the lateral width of the band 110. This may also allow for (or be provided to accommodate) curvature of the body part in the lateral direction (along the length of the body part) and accommodate lateral strain during body movement.

In some embodiments, the resiliently stretchable part of the band (extensible portion 112) may comprise multiple layers of fabrics and/or sheets/membranes to achieve the required elasticity for a given application. The resiliently stretchable part of the band (extensible portion 112) may comprise one layer, 2 layers, 3 layers, 4 layers, 5 layers, 6 layers, or more, for example.

The extensible portion 112 of different bands for different body parts may comprise different materials and/or numbers of layers of materials and/or thicknesses of materials to provide suitable elasticity. For example, in a compression band for a torso, there may be enough circumferential length in the band that a relatively high elasticity allowing a relatively high strain may be suitable, as there is enough circumferential distance available for relatively large spacing between indicator markings. In a compression band for a smaller body part, such as a forearm, there is not as much circumferential distance to work with, and so a relatively stiffer material (or less elastic material) may be required to achieve sufficient tension with a relatively smaller strain to adjust the compression band over a relatively shorter circumferential length of the band.

The indicator markings 130 may comprise any suitable marking or indicia for identifying the baseline circumference and target tension adjustment settings. For example, the indicator markings 130 may be printed onto the band 110, or stitched or woven into the band 110.

The indicator markings 130 could all be similar in appearance, with the user simply counting the markings to check which ones correspond to each other. Alternatively, the markings 130 may comprise different symbols, patterns or colours to differentiate them more easily.

Each of the markings 130 in each set of markings (131, 132, etc.) may include a visual similarity with the or each corresponding marking in the or each other set of markings.

For example, one of the second set of markings 132a may share a visual similarity with a corresponding one of the first set of markings 131a, which may have a similar shape, pattern, colour, or symbol. Further examples are illustrated in the embodiments shown in FIGS. 2A to 4E.

Referring to FIGS. 2A to 2E, an adjustable compression band 200 is shown, according to some embodiments. The compression band 200 includes similar features to compression band 100, and similar features are indicated with like reference numerals.

The compression band 200 comprises a flexible band 110 with a relatively elastic/extensible portion 112 and a relatively inelastic/inextensible portion 114 and an adjustment mechanism 120 (which may comprise part or all of the inextensible portion 114). The adjustment mechanism 120 comprises Velcro, but could comprise any other suitable adjustment mechanism as discussed above. The adjustment mechanism 120 further comprises a buckle 220 at the first end 101 and a Velcro tab 120 at the second end 102. The compression band 200 is fastened to the body part 1000 of a user as shown in FIGS. 2C and 2D by passing the Velcro tab 120 through a loop of the buckle 220, setting the tension, and folding the band 110 back on itself to fasten the Velcro tab 120 to the band 110 at the selected adjustment setting.

FIG. 2A shows a front side 212 of the compression band 200 which is configured to face away from the body part 1000 of a user when in use, as shown in FIGS. 2C, 2D, 2E. FIG. 2B shows a back side 214 of the compression band 200 which is configured to face towards the body part 1000 of the user when in use.

Depending on the type of adjustment mechanism, part of the front side 212 may be folded back on itself to face inward with the opposite part of the back side 214 facing outward, as shown in FIGS. 2D and 2E. Generally, the part of the front side 212 applying pressure to the body part is facing the body part, and the corresponding part of the back side 214 is facing away from the body part.

The indicator markings 130 comprise a first set of markings 231 on the front side 212, and second and third sets of markings 232, 233 on the backside 214.

The first set of markings 231 is used for determining the baseline circumference of the body part 1000, as shown in FIG. 2C. The second set of markings 232 includes corresponding markings for applying a first predetermined interface pressure, and the third set of markings 233 includes corresponding markings for applying a second predetermined interface pressure. As discussed above, the compression band 200 may comprise further sets of markings associated with other predetermined interface pressures.

Each set of markings 231, 232, 233 includes 5 markings associated with 5 different baseline circumferences (though any suitable number of circumferences may be marked and corresponding intermediate positions between markings may also be used for adjustment, e.g., the halfway point between 232a and 232b corresponds to the halfway point between 231a and 231b). Each marking in each set corresponds with a marking in each other set, as follows:
232a sets the first pressure and 233a sets the second pressure for baseline circumference 231a;
232b sets the first pressure and 233b sets the second pressure for baseline circumference 231b;
232c sets the first pressure and 233c sets the second pressure for baseline circumference 231c;
232d sets the first pressure and 233d sets the second pressure for baseline circumference 231d; and
232e sets the first pressure and 233e sets the second pressure for baseline circumference 231e.

The corresponding marks in each set are similar, with each marking, a, b, c, d, e, having a different number of dashes making up a broken line. As discussed above, the corresponding marks could be provided with other similarities, such as colours, shapes, or symbols, such as letters or numerals.

The different sets could also be distinguished with similarities within sets. For example, each set could have a different colour, such as 231 red, 232 blue, 233 black, and each corresponding marking having a similar symbol, A, B, C, D, E.

The compression band 200 can be used to apply a predetermined interface pressure to a body part 1000 of a user by first placing the compression band 200 around the body part 1000 of the user.

The circumference of the band 200 can then be adjusted to a neutral configuration as shown in FIG. 2C without applying significant pressure to the body part 1000 (i.e., with negligible tension in the band) to determine a baseline circumference of the body part 1000 indicated by the first set of indicator markings 231. The baseline circumference in FIG. 2C is indicated by marking 231c, for example.

The tension of the band 200 can then be adjusted to a target tension indicated by one of the second set of indicator markings corresponding to the baseline circumference. In FIG. 2D, the corresponding target tension indicator marking is 232c. If the second predetermined pressure is required, the tension can be adjusted to indicator marking 233c.

As discussed, any suitable number and types of markings can be used to cover different ranges of circumferences, different gradations, and different predetermined interface pressures or pressure profiles.

Referring to FIGS. 3A to 3C, an adjustable compression band 300 is shown according to some embodiments. The compression band 300 includes similar features to compression band 100, and similar features are indicated with like reference numerals.

The compression band 300 comprises a flexible band 110 with a relatively elastic/extensible portion 112 and a relatively inelastic/inextensible portion 114 and an adjustment mechanism 120 (which may comprise part or all of the inextensible portion 114). The adjustment mechanism 120 comprises eyelets 302 laced up with a cord or string 304 (e.g., like shoe laces or a corset). The eyelets may be hidden in a pocket or flap to avoid catching.

The compression band 300 may be formed as a continuous band in a loop which is slipped over the user's limb, for example. The band 300 is shown as if it were cut and laid flat in FIG. 3A. In some embodiments, it may be formed as a flat band with free ends laced together when worn by the user.

The indicator markings 130 of the compression band 300 are provided on separate indicator strips fixed to the band 110 at the ends. The first set of markings 331 is provided on a first indicator strip 310 and the second set of markings 332 is provided on a second indicator strip 320. The first and/or second indicator strips 310, 320 may be considered to form part of the adjustment mechanism 120.

Each strip 310, 320 may comprise a relatively elastic/extensible portion 312 and a relatively inelastic/inextensible portion 314. The extensible portion 312 may be relatively higher in elasticity than the extensible portion 112 of the band 110 so that the indicator strips 310, 320 have a negligible effect on the interface pressure applied by the compression band 300.

The first and second sets of markings 331, 332 are shown with corresponding numbers, but may be provided with any other corresponding sets of markings, as discussed above.

The compression band 300 can be used to apply a predetermined interface pressure to a body part 1000 of a user by first placing the compression band 300 around the body part 1000 of the user, as shown in FIG. 3B.

The circumference of the band 300 can then be adjusted to a neutral configuration as shown in FIG. 3B without applying pressure to the body part 1000 to determine a baseline circumference of the body part 1000 indicated by the first set of indicator markings 331. The baseline circumference in FIG. 3B is indicated by marking “3”, for example.

The tension of the band 300 can then be adjusted to a target tension indicated by one of the second set of indicator markings corresponding to the baseline circumference. In FIG. 3C, the corresponding target tension is also “3”.

The tension is adjusted by tightening the laces to draw the eyelets together (i.e., reducing the circumferential distance between the two sets of eyelets) and increase the tension of the band 110 with the indicator strips 310, 320 slipping over the top of the eyelets and the extensible portions 312 of the strips 310, 320 taking up the slack.

In other embodiments, different parts of the adjustment mechanism may comprise the indicator markings. For example, the indicator markings could be provided on the laces 304 and compared against one of the eyelets as a reference point. Alternatively, a tension adjustment system with a markings on a dial may be used to adjust the tension in the band. For example, the BOA fit system could be used to adjust tension in the laces to the target tension indicated on the dial.

In other embodiments, the compression band may comprise a substantially inextensible band with eyelets in the first and second ends laced together with an elastic cord configured to apply tension to the band. The indicator markings may be provided on the cord and compared against a reference on one of the eyelets. Alternatively, a tension adjustment system with a markings on a dial (e.g., BOA fit system) may be provided to adjust the cord to achieve the target tension required.

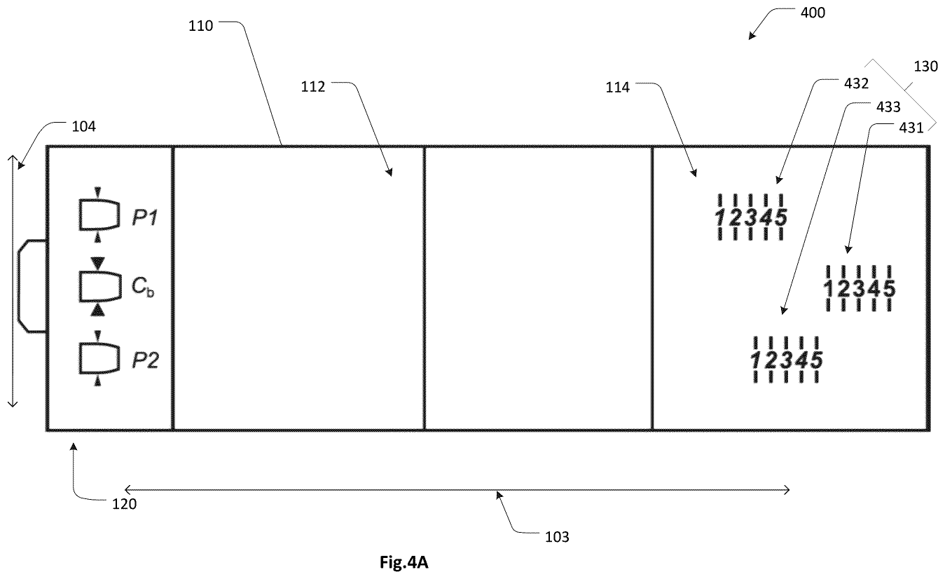

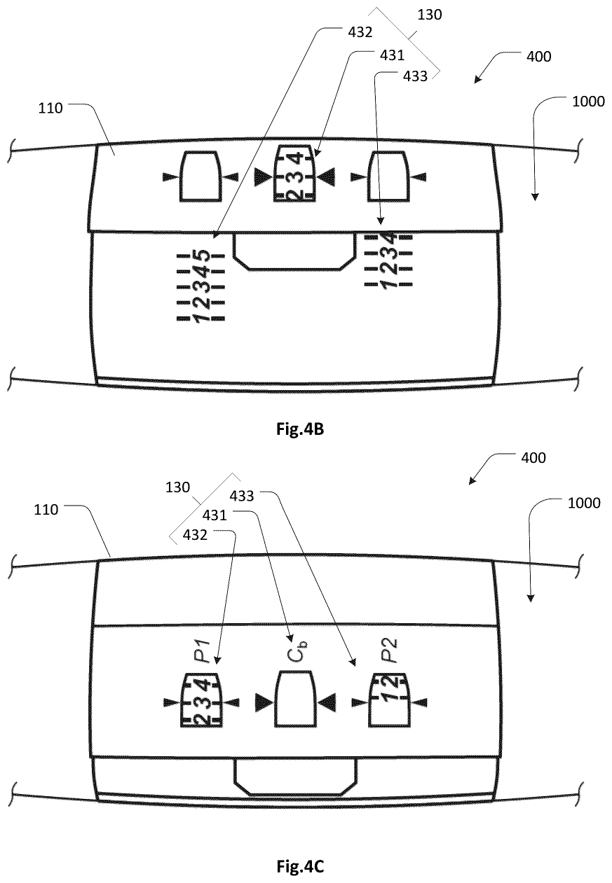

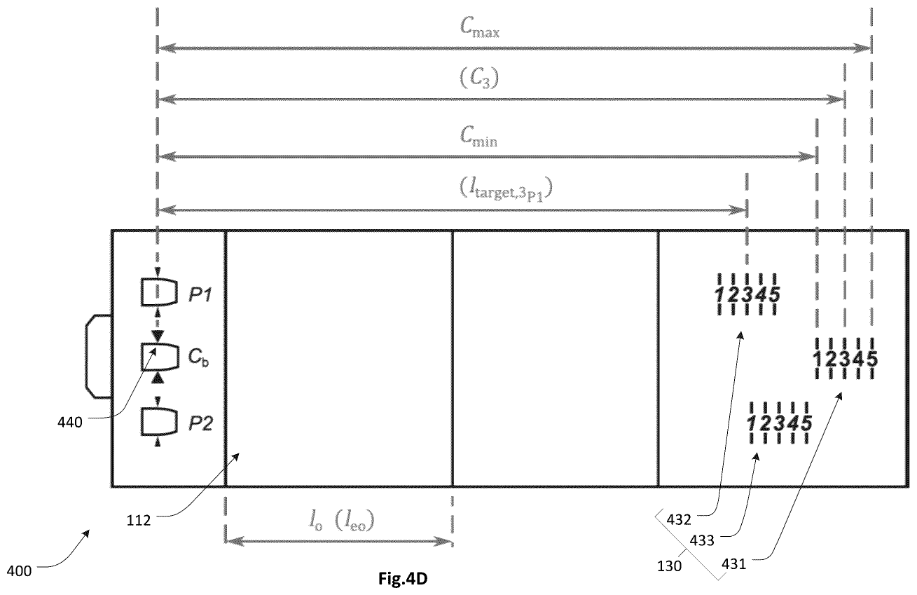

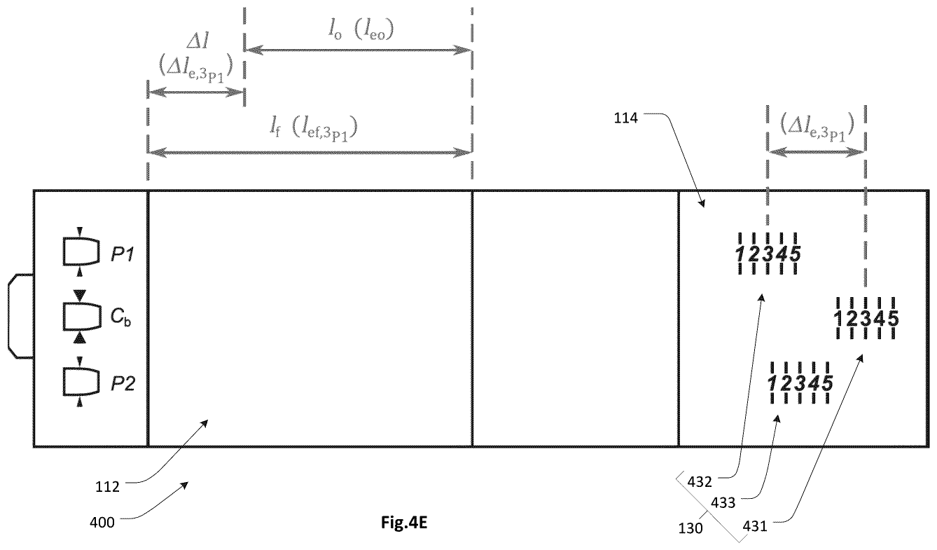

Referring to FIGS. 4A to 4C, an adjustable compression band 400 is shown according to some embodiments. The compression band 400 includes similar features to compression band 100, and similar features are indicated with like reference numerals.

The compression band 400 comprises a flexible band 110 with a relatively elastic/extensible portion 112 and a relatively inelastic/inextensible portion 114 and an adjustment mechanism 120 (which may comprise part or all of the inextensible portion 114). In this embodiment, the adjustment mechanism comprises Velcro tabs in a simple overlapping arrangement.

The indicator markings 130 are all provided on the front side, and arranged so that the overlapping tab at least partially overlaps the markings 130 when fastened. The overlapping tab also defines windows allowing the indicator markings 130 to be viewed when adjusted and fastened.

Alternatively, instead of providing windows for aligning with the indicator markings, the compression band 400 may be configured such that the leading edge of the band 110 is aligned with the indicator markings to determine the baseline circumference and set the tension to the corresponding target tension indicator markings.

The compression band 400 can be used to apply a predetermined interface pressure to a body part 1000 of a user by first placing the compression band 400 around the body part 1000 of the user, as shown in FIG. 4B.

The circumference of the band 400 can then be adjusted to a neutral configuration as shown in FIG. 4B without applying pressure to the body part 1000 to determine a baseline circumference of the body part 1000 indicated by the first set of indicator markings 431. The baseline circumference in FIG. 4B is indicated by marking “3”, for example.

The tension of the band 400 can then be adjusted to a target tension indicated by one of the second set of indicator markings 432 corresponding to the baseline circumference, as shown in FIG. 4C, with the corresponding target tension also shown as “3” for the second set of markings 432. Alternatively, the tension of the band 400 can be adjusted to a target tension indicated by one of the third set of indicator markings 433 corresponding to the baseline circumference, with the corresponding target tension also shown as “3” for the third set of markings 433.

Referring to FIGS. 4D and 4E, some dimensions are shown illustrating the location of certain indicator markings, and the following description sets out the calculations required to determine where the indicator markings should be provided along the length of the band in the circumferential direction.

In FIG. 4D, the baseline indicators (which may also be referred to as baseline circumference indicators and are referred to as the first set of markings 431 above) are shown aligned with the baseline circumference indicator window, marked (C_{b}).

The baseline indicators C₁, C₂, C₃, C₄, C₅, are marked as lines with numerals 1, 2. 3, 4, 5. As noted above, the markings could comprise any other symbols or visual coding, for example.

The baseline indicators may be built-in to (or otherwise disposed in or on) the compression band 400 to enable the ‘baseline circumference’ (C_{b}) of the local underlying body part to be determined at the time of donning and/or adjustment of the compression band 400.

The baseline indicators span a given range; that is, they encompass possible body part circumferences between a minimum circumference (Cₘᵢₙ) and a maximum circumference (Cₘₐₓ), as illustrated in FIG. 4D. The range of Cₘᵢₙ to Cₘₐₓ defines the working range of the adjustable section. As discussed previously, any suitable range and number of baseline circumferences may be included in the markings.

The circumferential location of a particular baseline circumference indicator C₃ is also illustrated with a dimension line (for example only) illustrating the circumferential distance from the baseline circumference indicator C₃ to an indexing mark 440 at the baseline circumference indicator window C_{b} in a neutral configuration (or rest state) without tension being applied to strain the band 110.

For any given baseline circumference C_{b}, there is a corresponding target tension (T_{target}) required to meet the predetermined target pressure (P_{target}), otherwise referred to as the predetermined interface pressure; the pressure to be applied to the body part.

As discussed above, the target tension may be indicated by the tension indicator, which may comprise target tension indicator markings (for example, the second and third sets of indicator markings 432, 433, each set associated with a different target tension).

Alternatively, as discussed above in relation to other embodiments, the tension in the band may be adjusted to a the target tension by monitoring another form of tension indicator, such as a tension meter, stress gauge, or strain gauge, for example.

In some embodiments, as shown in FIGS. 4A to 4E, the target tension indicator markings T_{target} are built-in to the garment to show the length of adjustment (i.e., strain of elastic elements) needed. The T_{target} indicators are pre-calculated (see equations below) and manufactured to be specific for a given predetermined pressure P_{target} given any current baseline circumference C_{b} of the underlying body part falling within the working range. More than one predetermined pressure P_{target} can be accommodated by having more than one set of target tension indicators T_{target}. For example, compression band 400 comprises a first set of target tension indicator markings (referred to above as the second set of markings 432) associated with a first predetermined pressure P₁, and a second set of target tension indicator markings (referred to above as the third set of markings 433) associated with a second predetermined pressure P₂.

As discussed above, each baseline circumference marking has a corresponding target tension indicator marking in each corresponding set, so that, for each baseline circumference, the user can adjust the tension to the corresponding target tension indicator marking to apply the first predetermined pressure P₁ or the second predetermined pressure P₂. The compression band 400 may include any suitable number of sets of markings, each associated with a corresponding target tension and predetermined pressure applied to the body part.

A reference point or index marking (e.g., viewing window, anchored marking, etc.) is provided for identifying the current reading from the baseline indicators and the T_{target} indicators. For example, the windows shown in FIG. 4D labelled C_{b}, P₁, P₂. Alternatively, the indicator markings could be positioned to correspond to the edge of the band at the first end of the band (i.e., distal from the markings). The reference point may align with the ‘true’ dimensions, or may have an offset that is accommodated during design and calculation.

The location of a particular target tension indicator marking is illustrated in FIG. 4D (in the neutral non-strain configuration) labelled 1_{target,3P1}. This marking is the target tension indicator marking for the first predetermined pressure P₁ associated with the third baseline circumference marking C₃.

With the compression band in place on the body (i.e., following donning), the two-step adjustment process is:
1. Determine C_{b}. The compression band is ‘neutrally’ tensioned around the underlying body part (to the neutral configuration) to remove the slack but without otherwise stretching the garment (at least not significantly). The C_{b} is then identified on the set of baseline indicators.
2. Adjust to the corresponding T_{target}. The compression band is ‘tightened’ (i.e., actively tensioned) on the body by stretching the garment and its elastic elements in the circumferential direction until the corresponding T_{target} is reached on the set of T_{target} indicators, then is fastened in place.

The compression band may be custom-made using the measured dimensions of the end user or may be generically made (manufactured without knowledge of the end user's dimensions). In either case, the mechanism described enables adjustment to achieve a given predetermined pressure P_{target}.

The calculations below can be used to determine the location of target tension indicator markings for a compression band (e.g., compression band 400) corresponding to a given set of baseline circumferences.

The compression band is modelled as a thin-walled pressure vessel, where the band 110 is considered to be representative of a pressure vessel wall. Following this approach, a free-body diagram of an open-ended cylindrical pressure vessel can be used to determine the local relationships between band stress in the circumferential direction, σ_{c} (N/m²), band thickness, t (m), pressure at the band-body interface, P (N/m²; normal to the body-band interface), and radius of a cross-sectional slice of the body part, r (m), which simplifies to:

$${σ}_{c} = \frac{P \cdot r}{c} \qquad ( 1 )$$

More generally, stress, σ, is a force, F, applied over and normal to an area, A, and given by:

$$σ = \frac{F}{A} \qquad ( 2 )$$

Considering a band section of a height, h (m), in the lateral direction 104, the band cross-sectional area, A_{b} (m²), relevant to σ_{c} is:

$${A}_{b} = t \cdot h \qquad ( 3 )$$

Considering the tension in the circumferential direction, Fc (N), and considering equations 2 and 3, σ_{c} can be expressed as:

$${σ}_{c} = \frac{{F}_{c}}{{A}_{b}} = \frac{{F}_{c}}{t \cdot h} \qquad ( 4 )$$

In some instances, it may be more convenient to consider a normalised circumferential tension in the band across the lateral width of the band, T_{c} (N/m), instead of σ_{c}. (i.e., normalised to the lateral width of the band). The normalised circumferential tension T_{c} can be expressed as:

$${T}_{c} = \frac{{F}_{c}}{h} \qquad ( 5 )$$

Combining equations 4 and 5 and rearranging gives:

$${σ}_{c} \cdot t = \frac{{F}_{c}}{h} = {T}_{c} \qquad ( 6 )$$

$${σ}_{c} = \frac{{T}_{c}}{t} \qquad ( 7 )$$

Combining equations 7 and 1 and rearranging gives:

$${σ}_{c} = \frac{{T}_{c}}{t} = \frac{P \cdot r}{t} \qquad ( 8 )$$

$${T}_{c} = P \cdot r \qquad ( 9 )$$

or, equivalently:

$$P = \frac{{T}_{c}}{r} \qquad ( 10 )$$

Thus, the advantage of using T_{c} (e.g., equation 9 or 10) rather than σ_{c} (e.g., equation 1) is that the material thickness becomes redundant in the case of using T_{c}.

For practicality, the body areas being compressed can be considered as an in-series assembly of adjacent cross-sectional slices. Accordingly, an estimate of r may be obtained by measuring the circumference of the corresponding body part, C (m), and calculating the mean radius, rₘ (m), of the cross-sectional slice represented by C. That is:

$${r}_{m} = \frac{c}{2 \cdot π} \qquad ( 11 )$$

For the purposes of manufacturing, sizing, and/or adjusting a compression band, the required P_{target} (N/m²) of a given band section can be substituted for P in the preceding equations. Thus, in combination with rₘ as an estimate of r, equation 9 becomes:

$${T}_{c} = {P}_{target} \cdot {r}_{m} \qquad ( 12 )$$

The force-strain relationship of the band and relevant constituent material(s) of the band (such as the resiliently stretchable part of the band) can be characterised (e.g., using a tensile tester) and expressed as tension-strain or stress-strain relationships (when force is first normalised to the material's lateral width or cross-sectional area, respectively). Continuing to work with the normalised tension (but also applicable to stress), the tension-strain relationship can then be utilised to calculate the strain required to produce a target tension. Assuming negligible strain in the inextensible portion if present, and a linear elastic relationship between tension, T (N/m), and strain, ϵ (unitless), the coefficient of proportionality, or elastic modulus (here in tension form cf. stress), E (N/m), is given as:

$$E = \frac{T}{ϵ} \qquad ( 13 )$$

Or, when rearranged to solve for strain:

$$ϵ = \frac{T}{E} \qquad ( 14 )$$

Considering now the compression band encircling a body section, the T_{c} calculated using equation 12 can be substituted for T and the strain of the elastic component in the circumferential direction, ϵ_{c} (unitless), can be substituted for ϵ, giving:

$${ϵ}_{c} = \frac{{T}_{c}}{E} \qquad ( 15 )$$

Combining equation 15 in turn with equations 12 and 11 gives:

$${ϵ}_{c} = \frac{{P}_{target} \cdot {r}_{m}}{E} \qquad ( 16 )$$

$$and$$

$${ϵ}_{c} = \frac{{P}_{target} \cdot ( \frac{c}{2 \cdot π} )}{E} \qquad ( 17 )$$

Where the tension-strain relationship is non-linear, a polynomial may be defined and used to solve for strain for a given tension, in place of the simple (linear) coefficient of proportionality given in equation 14. The non-linear variant follows the same logic as the process outlined here but may require stepped calculation (e.g., solve for T_{c} using equation 12, then estimate the ϵ_{c} required to attain that T_{c} using the polynomial) rather than the pooled calculation given in equation 17.

The baseline indicators are setup such that when the garment section being adjusted is neutrally tensioned, the C_{b} is identifiable. These baseline indicators may be marked on the garment in any form (numbers, symbols, colours, etc.) and by any means (stitching, printing, etc.).

The position of each baseline circumference indicator within the set corresponds to an actual (known) circumferential length. The baseline indicators span a working range from a given Cₘᵢₙ to a Cₘₐₓ, as shown in FIG. 4D. The compression band may be custom made or selected from generically made options to ensure the end-user's C_{b} and (if relevant) any anticipated change in C_{b} with time falls within the working range.

For purposes of calculation and manufacture, all individual indicators representing the working range of Cₘᵢₙ to Cₘₐₓ are indexed for a given band section. If the nth baseline circumference indicator within the indexed set from Cₘᵢₙ to Cₘₐₓ is specified generally as Cₙ (m), then the nth corresponding strain, ϵ_{c,n} (unitless), required for each Cₙ to meet the given P_{target} (and therefore T_{target}) can be calculated in turn by substituting ϵ_{c,n} for ϵ_{c} and Cₙ for C in equation 17 (or the non-linear equivalent, if a polynomial is used); regarding the linear variant, that is:

$${ϵ}_{c , n} = \frac{{P}_{target} \cdot ( \frac{{c}_{n}}{2 \cdot π} )}{E} \qquad ( 18 )$$

Strain is also defined as:

$$ϵ = \frac{Δ l}{{l}_{o}} \qquad ( 19 )$$

where lₒ (m) is the original length (of the resiliently stretchable portion in the neutral configuration, FIG. 4D) and Δl (m) is the change in length (after stretching under the target tension to the corresponding strain, FIG. 4E). The final length is l_{f} (m), as shown in FIG. 4E, and the change in length, Δl is:

$$Δ l = {l}_{f} - {l}_{o} \qquad ( 20 )$$

Where the band comprises elastic and inelastic materials in the circumferential direction (extensible and inextensible parts), then the strain during band adjustment is assumed to occur principally in the elastic section and, therefore, the elastic section is characterised by E (equation 13) or its non-linear equivalent (polynomial). The original elastic length, lₑₒ (m), is specified during design of the compression band (i.e., is known) so, accordingly, the change in length of the extensible portion, Δl_{e,n} (m), required to meet the target ϵ_{c,n} can be calculated by substitution into equation 19 and rearranging as:

$${ϵ}_{c , n} = \frac{Δ {l}_{e , n}}{{l}_{e o}} \qquad ( 21 )$$

$$Δ {l}_{e , n} = {ϵ}_{c , n} \cdot {l}_{e o} \qquad ( 22 )$$

Therefore, with the circumference of the underlying body part assumed to remain unchanged during garment adjustment itself, and ϵ_{c,n} being the strain required of the elastic element in the ‘final’ position (i.e., when adjusted on the body), the position of the nth T_{target} indicator, l_{target,n} (m), that corresponds to the nth baseline circumference indicator (i.e., Cₙ) can be calculated as:

$${l}_{target , n} = {C}_{n} - Δ {l}_{e , n} \qquad ( 23 )$$

The calculation process detailed above so far assumes a generally uniform C, T_{c}, and, therefore P with h for a given band section (i.e., elastic and inelastic sections are rectangular); in this case, the elastic component's lₒ, l_{f}, and Δl during adjustment will all also be consistent with h. This assumption is reasonable where the underlying body part is sufficiently cylindrical, which may be achieved where h is inversely proportional to the local longitudinal curvature (e.g., use of a smaller h where the longitudinal curvature is greater, and vice versa).

The process of calculation is repeated for each set of indicators for each desired P_{target} (P1, P2, etc.) Accordingly, each P_{target} will have its own set of T_{target} indicators but will work off the one set of baseline indicators.

In use there may be small changes in circumference of the body part due to compression. These changes are assumed to be negligible in the above model, but could be accounted for, if required.

As discussed above, in some embodiments, the band may be non-rectangular (and may vary in circumference across the lateral width and/or vary in lateral width along the circumference) to allow modulation of local T_{c} with h. Cases where non-rectangular bands may be used include where a uniform pressure is desired but the body part is not sufficiently cylindrical (i.e., body C varies with h across the lateral width of the band), or where a non-uniform pressure profile across the lateral width of the band is desired with or without sufficiently cylindrical body sites (i.e., P_{target} varies with h).

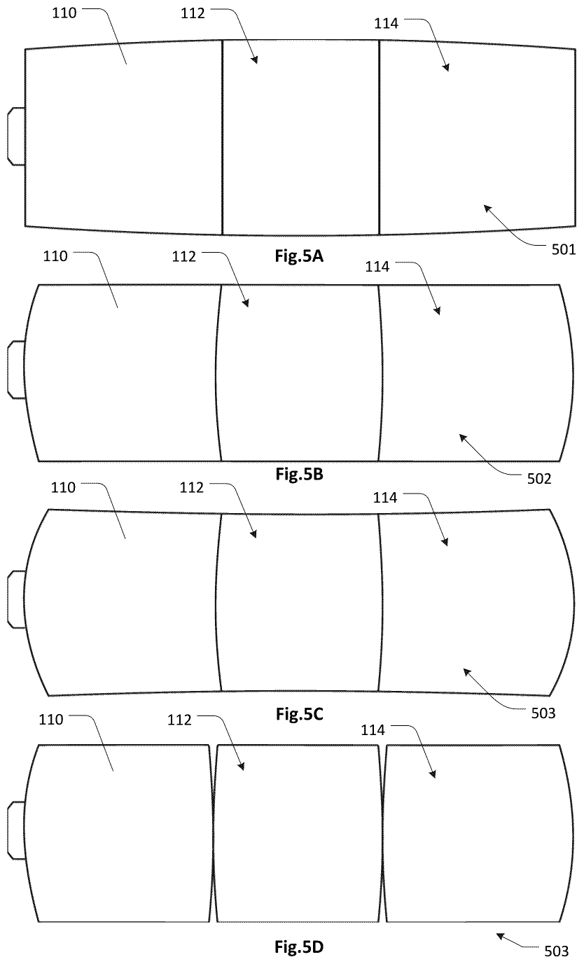

Some examples of different band shapes are set out in FIGS. 5A, 5B, 5C and 5D. The band 501 in FIG. 5A has a uniform circumference across the lateral width of the band, and the lateral width of the band varies along the circumference. The band 502 in FIG. 5B varies in circumference across the lateral width of the band, and the lateral width of the band is constant along the circumference. The band 503 in FIG. 5C varies in circumference across the lateral width of the band, and varies in lateral width along the circumference of the band.

Non-rectangular bands may be manufactured according to any suitable method. FIG. 5D illustrates one suitable method for manufacturing non-rectangular bands, such as band 503 of FIG. 5C. That is, by stitching together flat pieces of fabric with curved edges to create curvature in the compression band, as often done in tailoring for fashion garments. Alternatively, other tailoring techniques may be used, such as sewing in darts, for example.

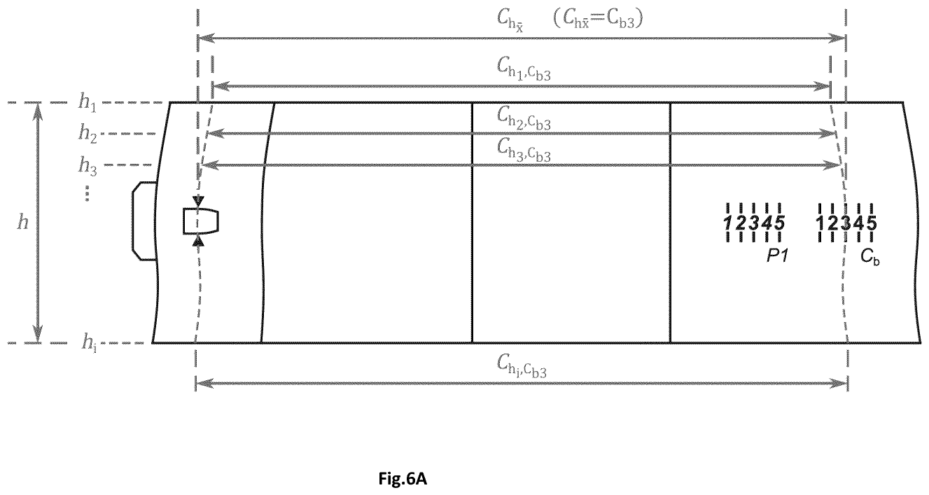

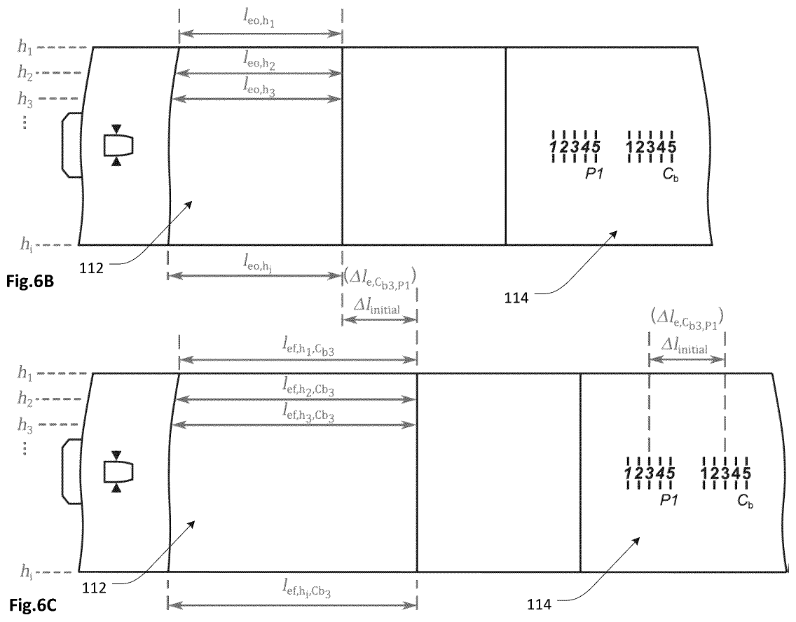

For a given non-rectangular band, the elastic component's lₒ and l_{f} differs with h while the Δl during adjustment is itself typically consistent with h as shown in FIGS. 6A, 6B, 6C. Therefore, the outcome for shaped sections is that the e of the elastic component also differs with h. For this reason, the Δl of the shaped section needs to be considered when determining the elastic component's lₒ (cf. rectangular garment sections, where the lₒ can be more freely chosen).

Where the end user's body dimensions have been measured for design and manufacture of the compression band, the process of calculation described below is based on those corresponding body measurements. The band is indexed in the direction of h (h₁₋ᵢ), as shown in FIGS. 6A, 6B, 6C.

At each corresponding indexed position spanning h₁₋ᵢ, a local circumference measurement (Cₕ₁₋ᵢ; unit m) and the desired local P_{target}, P_{target,h1-i (unit N/m}²), is specified. Each of P_{target,h1-i} may be the same across all sites (uniform pressure with h) or may be different (e.g., progressive or degressive pressure with h) to create a non-uniform pressure profile across the lateral width of the band. At each h₁₋ᵢ, the target T_{c} (T_{c,h1-i}; unit N/m; equations 11 and 12) and corresponding ϵ_{c} (ϵ_{c,h1-i}; unitless; equation 15 or non-linear equivalent) required to achieve each P_{target,h1-i} are calculated.

For the step of establishing the initial Δl required for the whole band to achieve each of P_{target,h1-i} with original body dimensions, the variable values spanning the positions of h₁₋ᵢ are pooled. Accordingly, the mean of all Cₕ₁₋ᵢ, C_{hx̅} (unit m), is used to calculate a single mean radius, r_{hx̅} (unit m). In combination with the mean of all P_{target,h1-i}, P_{target,hx̅} (unit N/m²), the representative band-section T_{c}, T_{c,section} (unit N/m), is then calculated (again following equations 11 and 12). The section ϵ_{c}, ϵ_{c,section} (unitless), required to achieve T_{c,section} (equation 15 or non-linear equivalent) is used to calculate the initial Δl for the band section, Δlᵢₙᵢₜᵢₐₗ (unit m), by specifying a suitable placeholder lₑₒ (equation 22).

The Δlᵢₙᵢₜᵢₐₗ is then used to calculate the length of the elastic section in the circumferential direction at each h₁₋ᵢ. That is, using Δlᵢₙᵢₜᵢₐₗ with each ϵ_{c,h1-i}, the specific lₑₒ at each position of h₁₋ᵢ, l_{eo,h1-i} (m), can be calculated in turn as:

$${l}_{e o , {h}_{i}} = \frac{Δ {l}_{initial}}{{ϵ}_{c , {h}_{i}}} \qquad ( 24 )$$

Thus, the process above gives the dimensions of the elastic section for manufacture and gives the strain required to achieve target pressures (i.e., all P_{target,h1-i}) at the initial body dimensions.

Because C inherently varies with h for shaped sections, the baseline indicators and T_{target} indicators may be positioned on the band where the local C is representative of the garment section mean (i.e., local C approximates C_{hx̅}).

The baseline indicators for shaped garment sections may be indexed in the appropriate direction(s) from the C_{hx̅} (i.e., in accordance with what body anthropometric changes are intended to be accommodated). The T_{target} indicators can be calculated from the indexed C (i.e., Cₙ) in the same way as for rectangular sections (see equations 18-23), except that P_{target,hx̅} is used in place of P_{target}.

The band shown in FIGS. 6A to 6C illustrates that the various sets of indicator markings may be aligned with each other in the lateral direction and vary in location in the circumferential direction as set out in the calculations above. Similarly, in the other embodiments described above, the sets of indicator markings may be aligned with each other in the lateral direction. In some embodiments, the various sets of indicator markings may be offset from each other in the lateral direction.

The principles set out above may be used to design and manufacture compression bands for many different suitable applications covering a wide range of shapes and dimensions to suit different body parts and users; different therapeutic, prophylactic, or performance applications; and different ranges of interface pressures for different applications.

Some examples are provided below for illustrative purposes only, and other embodiments of the compression band may comprise any other suitable dimensions and be configured to apply any other suitable pressures or pressure profiles for other applications.

For various body parts in different scenarios, a range of baseline circumferences is provided as well as a corresponding range of circumferential tension (normalised to the lateral width of the band) required to apply the corresponding range of interface pressures to the body part.

### Examples 1 and 2—Space: Mechanical Counterpressure (MCP) Extravehicular Activity (EVA) Suits and Orthostatic Intolerance Garments (OIG)

A first example (Table 1) gives ranges for a MCP EVA suit using a uniform 180 mmHg (24 kPa) as an example minimum pressure and a uniform 300 mmHg (40 kPa) as an example maximum pressure.

A second example (Table 2) gives ranges for an OIG with a pressure profile graduating from 55 mmHg at the ankle, to 35 mmHg at the knee, to 18 mmHg at the proximal thigh, and a uniform 16 mmHg over the abdomen.

The corresponding body site circumferences used in Table 1 and Table 2 are from the anthropometric dataset provided in NASA's Human Integration Design Handbook [NASA, Human Integration Design Handbook. NASA/SP-2010-3407/Revision 1. Washington, DC: National Aeronautics and Space Administration (NASA), 2014] except for the ankle circumference which is estimated below.

This anthropometric dataset specifies a size range of an astronaut population (extrapolated to the year 2015) for specific body sites, encompassed by minima (typically 1st-percentile female) and maxima (typically the 99th percentile male) values. An ankle circumference was not reported, so proportionate estimates were used instead. The normalised circumferential tension minima are calculated using the minimum pressure and the minimum circumference for each body site, and the normalised circumferential tension maxima are calculated using the maximum pressure and the maximum circumference for each body site.

TABLE 1
Example value ranges for a mechanical counterpressure
extravehicular activity suit at specific body sites.

| | Pressure | Circumference | Circumferential |
|---|---|---|---|
| Body site | (mmHg) | (mm) | tension (N/m) |
| Chest (230) | 180-300 | 757-1186 | 2891-7550 |
| Waist (931) | 180-300 | 612-1105 | 2337-7034 |
| Thigh (852) | 180-300 | 478-719 | 1826-4577 |
| Calf (207) | 180-300 | 295-445 | 1127-2833 |
| Biceps, flexed (111) | 180-300 | 229-404 | 875-2572 |
| Forearm, flexed (369) | 180-300 | 216-353 | 825-2247 |
| Wrist (967) | 180-300 | 135-198 | 516-1260 |

TABLE 2
Example value ranges for an orthostatic intolerance
garment at specific body sites.

| | Pressure | Circumference | Circumferential |
|---|---|---|---|
| Body site | (mmHg) | (mm) | tension (N/m) |
| Waist (931) | 16 | 612-1105 | 208-375 |
| Thigh (852) | 24 | 478-719 | 243-366 |
| Calf (207) | 40 | 295-445 | 250-378 |
| Ankle (estimated) | 55 | 174-280 | 203-327 |

### Example 3—Thigh-Length Medical Compression Hosiery (Graduated Profile)

A third example (Table 3) gives ranges for thigh-length medical compression hosiery, following the compression class definitions, site locations, and graduated pressure profiles given in [Deutsches Institut für Gütesicherung und Kennzeichnung, Medical Compression Hosiery. Quality Assurance RAL-GZ 387/1. Sankt Augustin: RAL Deutsches Institut für Gütesicherung und Kennzeichnung e.V., 2008].

The circumferential tension minima are calculated using the minimum pressure and the minimum circumference for each body site, and the circumferential tension maxima are calculated using the maximum pressure and the maximum circumference for each body site.

TABLE 3
Example value ranges for thigh-length
medical compression hosiery.

| | Pressure | Circumference | Circumferential |
|---|---|---|---|
| Compression class and site | (mmHg) | (mm) | tension (N/m) |
| Class I | | | |
| Proximal thigh (G) | 3.6-12.6 | 430-800 | 33-214 |
| ~Mid-thigh (F) | 3.6-12.6 | 350-710 | 27-190 |
| Calf at maximum girth (C) | 9.0-16.8 | 260-480 | 50-171 |
| Proximal ankle (B1) | 12.6-21.0 | 190-400 | 51-178 |
| Ankle at minimum girth (B) | 18.0-21.0 | 150-330 | 57-147 |
| Class II | | | |
| Proximal thigh (G) | 4.6-16.0 | 430-800 | 42-272 |
| ~Mid-thigh (F) | 4.6-16.0 | 350-710 | 34-241 |
| Calf at maximum girth (C) | 11.5-25.6 | 260-480 | 63-261 |
| Proximal ankle (B1) | 16.1-32.0 | 190-400 | 65-272 |
| Ankle at minimum girth (B) | 23.0-32.0 | 150-330 | 73-224 |
| Class III | | | |
| Proximal thigh (G) | 6.8-18.4 | 430-800 | 62-312 |
| ~Mid-thigh (F) | 6.8-18.4 | 350-710 | 51-277 |
| Calf at maximum girth (C) | 17.0-32.2 | 260-480 | 94-328 |
| Proximal ankle (B1) | 23.8-46.0 | 190-400 | 96-390 |
| Ankle at minimum girth (B) | 34.0-46.0 | 150-330 | 108-322 |
| Class IV | | | |
| Proximal thigh (G) | 9.8-26.0 | 430-800 | 89-441 |
| ~Mid-thigh (F) | 9.8-26.0 | 350-710 | 73-392 |
| Calf at maximum girth (C) | 24.5-45.5 | 260-480 | 135-463 |
| Proximal ankle (B1) | 34.3-65.0 | 190-400 | 138-552 |
| Ankle at minimum girth (B) | 49.0-65.0 | 150-330 | 156-455 |

As discussed previously, the adjustable compression bands described herein may be provided and used as separate devices, or in some cases, may form part of a garment comprising one or more adjustable compression bands.

In some embodiments, a plurality of adjustable compression bands may be combined into a garment with non-adjustable portions between them. For example, the non-adjustable portions may be compression or non-compression portions.

In some embodiments, a plurality of adjustable compression bands may be combined adjacent to each other to create a garment or part of a garment such as a sleeve. The bands may be manufactured separately and then joined together, such as by stitching and/or adhesive, for example. Alternatively, the bands may be integrally formed with each other.

FIGS. 7A and 7B illustrate a garment 700 (or sleeve) for a lower leg comprising a plurality of adjustable compression bands 701. Each compression band 701 may comprise any one or more of the features described in relation to the other embodiments.

The bands 701 are integrally formed with each other in that a large common band 710 is provided extending across the lateral width of all of the bands 701 combined and connecting the bands 701 to each other. Each band 701 may comprise independent extensible/elastic portions 712 separated by slits.

The common band 710 may comprise an inextensible portion 714, which may comprise the entirety of the common band 710. In other embodiments, the common band 710 may comprise an extensible portion 712 as well as an inextensible portion 714. In some embodiments, the extensible portion 712 of the common band 710 may comprise the extensible portion (or part thereof) of each of the bands 701.

Each band 701 includes an independent adjustment mechanism 720 (e.g., a hook-and-loop fastener such as Velcro) with slits between them. The adjustment mechanism 720 of each band 701 may be wrapped around the body part (e.g., lower leg) and fastened to the inextensible portion 714 according to indicator markings as described above (not shown in FIGS. 7A and 7B). The inextensible portion 714 and indicator markings may be considered part of the adjustment mechanism 720.

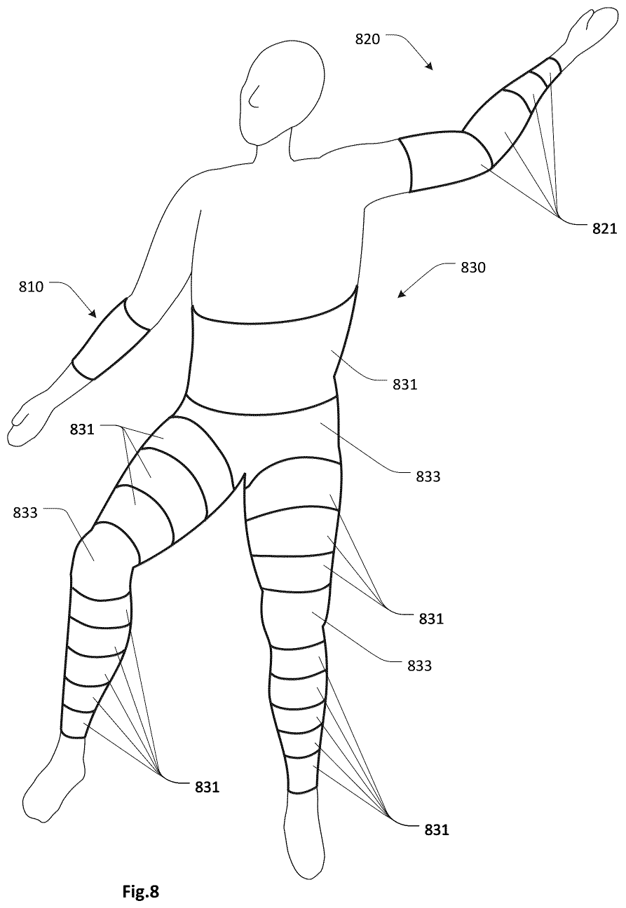

Referring to FIG. 8, three other garments comprising adjustable compression bands are illustrated, for example only.

Garment 810 is a partial sleeve for the forearm. Garment 810 may comprise a plurality of compression bands, which may be integrally formed as described in relation to garment 700 or formed separately and joined together. Alternatively, garment 810 may comprise a single adjustable compression band according to any one of the described embodiments.

Garment 820 is another sleeve configured to extend above the elbow from the wrist. Garment 820 comprises a plurality of adjustable compression bands 821. The compression bands 821 are illustrated as being formed separately and joined together, but could alternatively be integrally formed, as described above.

Garment 830 is a partial body suit extending from the ankles up to cover the lower abdomen. Garment 830 comprises a plurality of adjustable compression bands 831, some of which are joined directly to each other, and others of which are indirectly joined to each other by non-adjustable portions 833 to form the garment 830. For example, garment 830 may include non-adjustable elastic portions to allow better mobility/flexibility in joints such as around the knees and hips.

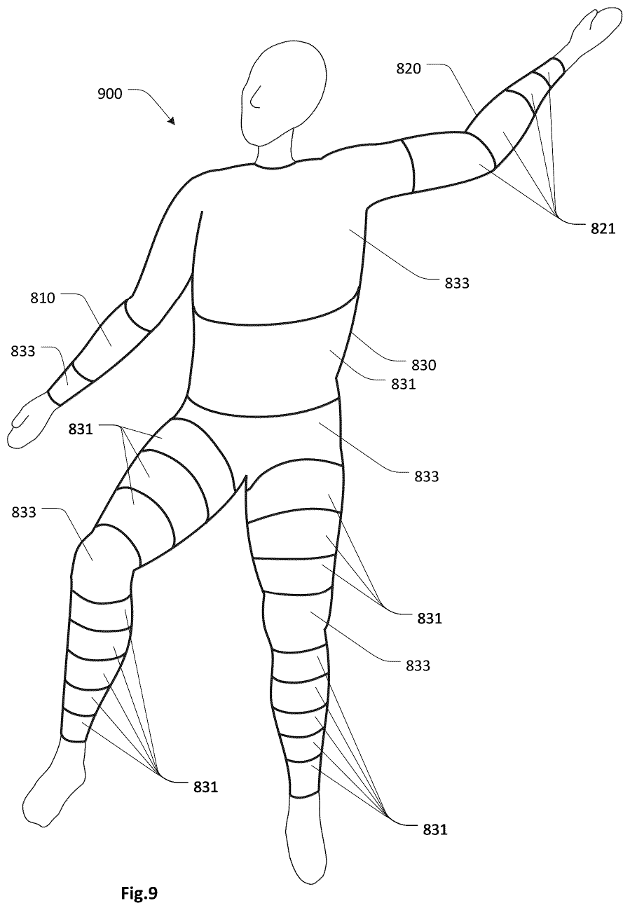

As discussed above, the compression bands may be combined in many different ways and integrated into various garments for different applications. For illustrative purposes only, FIG. 9 shows garment 900 which includes additional non-adjustable portions 833 to combine the garments 810, 820, 830 of FIG. 8 into a single garment 900; a full-body suit. Any other suitable combinations of compression bands and garment configurations may be implemented for different applications.

Additional embodiments of the compression band are described below, for illustrative purposes, any of which may be used separately or incorporated into a garment as described above.

As described above, the band may comprise any suitable shape, and may be rectangular or may vary in circumferential length across the width of the band, or may vary in lateral width along the circumference of the band. The side edges of the band may be straight or curved, and may be concave or convex or a combination of concave and convex.

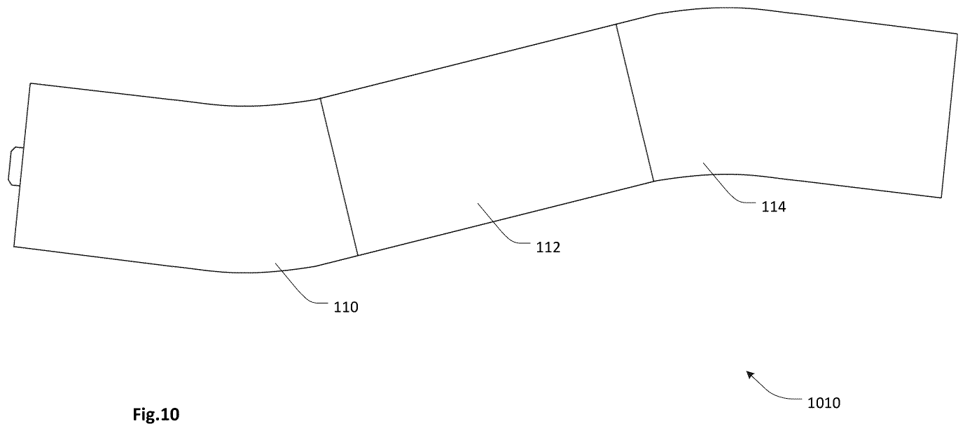

Referring to FIG. 10, an alternative shaped band 1010 is shown for illustrative purposes. The band 1010 is shown as following an S-shape, with the side edges following a curved path while remaining substantially parallel to each other. In some embodiments, the lateral width of the band may be constant along the circumferential length while defining curved edges.

In some embodiments, part of one edge may be curved to account for anatomy around a particular body part. For example, for a band configured to provide compression for the upper thigh, the top edge of the band may dip down around the crotch while raising higher around the outer part of the thigh.

More generally, the band may comprise any suitable shape including curved or contoured shapes to fit different body parts and/or to apply a particular interface pressure profile along the body part.

Referring now to the precision of indicator markings and tension settings.

While in some embodiments the target tension indicator markings may be provided on the resiliently stretchable part of the band, this may introduce some error or uncertainty in the precise position of the markings and the corresponding tension in the band. For example, in having to account for the change in spacing of the markings at different levels of strain in the band when calculating the locations of the markings, as well as the precision of the markings themselves on the stretchable portion, which may deform slightly under strain.

Therefore, it may be beneficial to provide the target tension indicator markings on a relatively inextensible part of the adjustment mechanism. Various embodiments incorporating this feature are set out below in addition to the embodiments set out above describing the target tension indicator markings on the relatively inextensible part of the band.

Some embodiments relate to an adjustable compression band comprising: a band of flexible material configured to extend circumferentially around a body part of a user, at least part of the band being resiliently stretchable in the circumferential direction; a baseline circumference indicator for determining a baseline circumference of the body part; and an adjustment mechanism for adjusting a tension of the band in the circumferential direction to apply pressure to the body part, wherein the adjustment mechanism comprises an inextensible element, which is relatively inextensible in comparison with the resiliently stretchable part of the band, and which comprises a tension indicator for indicating a target tension adjustment setting corresponding to the determined baseline circumference, the target tension adjustment setting corresponding to a tension required for the band to apply a predetermined interface pressure to the body part based on the determined baseline circumference.

In some embodiments, the inextensible element also comprises the baseline circumference indicator.

With reference to the embodiments described above and below, similar reference numerals refer to similar or corresponding features.

In some embodiments, the inextensible element comprises a relatively inextensible part of the band with the tension indicator comprising markings on the relatively inextensible part of the band.

For example, with reference to FIGS. 1, 2A-2E, 4A-4E, 5A-5D, 6A-6C, the adjustment mechanism 120 may also be considered to comprise the inextensible portion 114 of the band (which may be considered an inextensible element as described above) with the baseline markings and target tension indicator markings.

With reference to FIGS. 3A-3C, the adjustment mechanism 120 may also be considered to comprise the inextensible portions 314 of the indicator strips (which may be considered an inextensible element as described above) with the markings 331, 332.

With reference to FIGS. 7A-7B, the adjustment mechanism 720 may also be considered to comprise the inextensible portion 714 (which may be considered an inextensible element as described above) and the indicator markings on the inextensible portion 714.

Other embodiments incorporating an inextensible portion of the band as the inextensible element of the adjustment mechanism may include any other suitable tensioners and/or fasteners with target tension indicator markings incorporated into the inextensible portion of the band. For example, whether the band has overlapping free ends, as shown in FIGS. 4A-4E, or a free end passing through a buckle and folding back on itself, as shown in FIGS. 1 and 2A-2E, the compression band may comprise a plurality of fasteners positioned at different locations along the circumferential length of the inextensible portion of the band, so that the free end of the band may be attached to the fasteners at a selected circumferential length corresponding to a selected target tension setting. In some embodiments, the target tension indicator markings may comprise the fasteners. For example, the fasteners may comprise rows of eyelets, each set at a particular circumferential length corresponding to a particular tension setting, and configured to engage a corresponding row of hooks on the free end of the band. In other embodiments, the fasteners may comprise an array of zipper halves, each set at a particular circumferential length corresponding to a particular tension setting, and configured to engage a corresponding zipper half on the free end of the band.

In some embodiments, the inextensible element comprises a relatively inextensible cord coupling ends of the band together with the tension indicator comprising markings on the inextensible cord. For example, laced up eyelets with a cord or string laced through the eyelets to couple different portions of the band together and adjust and fix the tension of the band.

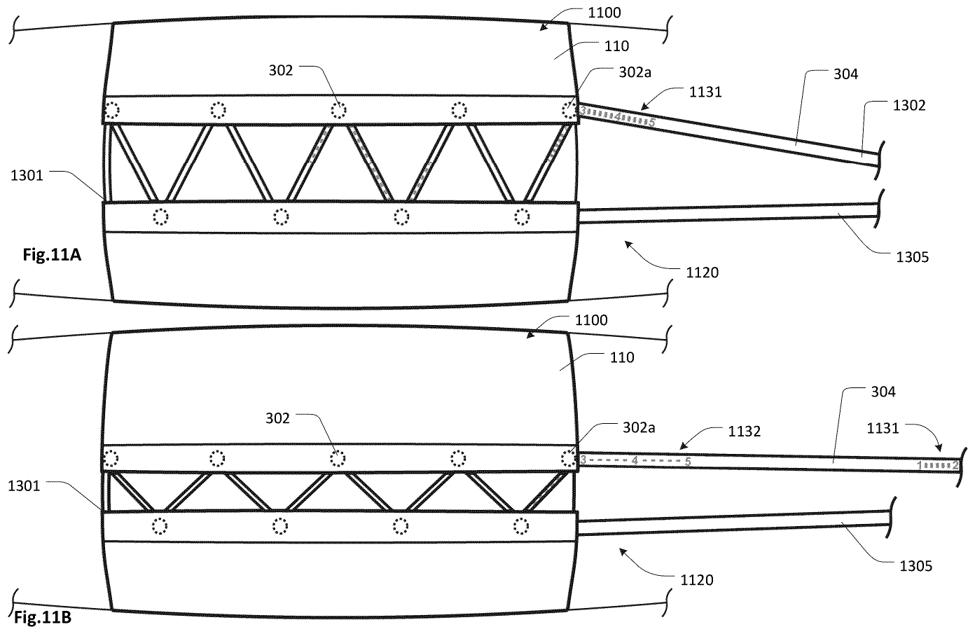

Referring to FIGS. 11A and 11B, a compression band 1100 is shown, according to some embodiments, illustrating an adjustment mechanism 1120. The compression band comprises a band 110 shown wrapped around a limb of a user. The band comprises a resiliently stretchable portion and may or may not also include a relatively inextensible portion of the band 110. The portions of the band 110 shown in the FIG. 10 may be resiliently stretchable or relatively inextensible portions.

The adjustment mechanism 1120 comprises eyelets 302 laced up with a cord or string 304 (e.g., like shoe laces or a corset). The eyelets may be hidden in a pocket or flap to avoid catching. The cord/string/lace may comprise any suitable material and configuration, such as twisted, braided or monofilament wire, for example.

A fixed end 1301 of the cord is fixed to part of the band 110 and a free end 1302 of the cord is laced through the eyelets 302 and configured to be pulled to adjust the tension in the band 110. The free end 1302 may be fastened to part of the band 110 to set the tension. For example, the free end 1302 may be tied to a tie-off point such as a second fixed cord 1305, or any other suitable tie-off point, such as a hook or eyelet.

The baseline circumference indicator markings 1131 and target tension indicator markings 1132 are marked on the cord 304. The markings 1131, 1132 are visible as the cord 304 is pulled through the eyelets 302.

The baseline circumference can be determined by tensioning the cord 304 and tightening the band 110 to a neutral configuration and reading the corresponding baseline circumference marking 1131 visible at the last eyelet 302a where the free end 1302 of the cord leads away from the band 110. Then the predetermined interface pressure may be applied by further tensioning the cord 304 until the target tension marking 1132 corresponding to the determined baseline circumference marking 1131 is visible at the last eyelet 302a. The cord 304 can then be tied off under tension to maintain the target tension in the band 110.

The cord 304 is relatively inextensible compared with the resiliently stretchable portion of the band 110. This improves the precision of the measurement of the baseline markings and the setting of the target tension compared with markings on the resiliently stretchable part of the band.

In some embodiments, the adjustment mechanism 120 may comprise laced up eyelets, and the inextensible element may comprise a relatively inextensible part of the band with indicator markings configured such that the baseline circumference and target tension markings can be measured based on the free end of the cord 304. Again, the relatively inextensible nature of the cord and the relatively inextensible part of the band with the indicator markings improve the precision of the measurement compared with markings on the resiliently stretchable part of the band.

In some embodiments, the adjustment mechanism comprises a ratcheting tensioner and the inextensible element comprises a tensioning dial of the tensioner with the tension indicator comprising markings on the dial. For example, the ratcheting tensioner may comprise a reel configured to take up part of a lacing cord (laced through eyelets) and rotate to increase the tension via a ratcheting mechanism controlled by a tensioning dial. The tensioning dial may be relatively rigid with the target tension indicator markings marked on the dial relative to a reference point, similar to the Boa fit system. Again, this allows for a more precise definition of the target tension settings, to improve the precision of the measurement compared with markings on the resiliently stretchable part of the band. In such embodiments, the baseline circumference indicator markings may be provided on another part of the band or on part of the cord, for example, as described above.

In all embodiments described above, the baseline circumference indicator markings may be provided on the inextensible element to improve precision, or alternatively, on the resiliently stretchable part of the band. As there is negligible strain on the resiliently stretchable part of the band during determination of the baseline circumference, it may not have much impact as to whether the baseline circumference indicator markings are provided on the inextensible element or not.

An issue that may introduce errors into determination of the baseline circumference is how precisely the neutral configuration for determining the baseline circumference is defined. As described above, the baseline circumference should be determined in a neutral configuration with a snug fit and negligible tension in the band. In order to improve the precision of the baseline circumference determination, a baseline tension may be defined, which the band should be set to when determining the baseline circumference.

The baseline tension may be selected to correspond to a relatively small applied interface pressure, such as at least 0.5 mmHg, at least 1 mmHg, at least 2 mmHg, at least 3 mmHg, less than 5 mmHg, less than 3 mmHg, less than 2 mmHg, in the range of 0.5 mmHg to 5 mmHg, in the range of 1 mmHg to 3 mmHg, about 1 mmHg, about 2 mmHg or about 3 mmHg, for example.

Table 4 shows a range of normalised circumferential tension (normalised across the width of the band) for achieving applied interface pressures in the range of 1 mmHg to 3 mmHg for various body parts and circumferences. The corresponding body site references and circumferences shown in Table 4 are from the anthropometric dataset provided in NASA's Human Integration Design Handbook [NASA, Human Integration Design Handbook. NASA/SP-2010-3407/Revision 1. Washington, DC: National Aeronautics and Space Administration (NASA), 2014]

TABLE 4
Example value ranges for the baseline
tensioner at specific body sites.

| | Pressure | | |
|---|---|---|---|
| | equivalent | Circumference | Circumferential |
| Body site | (mmHg) | (mm) | tension (N/m) |
| Chest (230) | 1-3 | 757-1186 | 16-75 |
| Waist (931) | 1-3 | 612-1105 | 13-70 |
| Thigh (852) | 1-3 | 478-719 | 10-46 |
| Calf (207) | 1-3 | 295-445 | 6-28 |
| Biceps, flexed (111) | 1-3 | 229-404 | 5-26 |
| Forearm, flexed (369) | 1-3 | 216-353 | 5-22 |
| Wrist (967) | 1-3 | 135-198 | 3-13 |

The baseline tension may be set at different levels depending on the application and/or on the relevant body part/location. Any suitable baseline tension may be defined, such as a normalised circumferential tension in the range of 1 N/m to 100 N/m, 3 N/m to 75 N/m, 1 N/m to 10 N/m, 10 N/m to 50 N/m, 5 N/m to 50 N/m, 5 N/m to 25 N/m, less than 100 N/m, less than 80 N/m, less than 50 N/m, less than 25 N/m, less than 10 N/m, at least 1 N/m, at least 2 N/m, at least 3 N/m, at least 5 N/m, at least 10 N/m, about 1 N/m, about 2 N/m, about 3 N/m, about 5 N/m, about 10 N/m, or about 20 N/m, for example.

In some embodiments the compression band, further comprises a baseline tension indicator configured to indicate when a predetermine threshold baseline tension is applied to the band, the baseline tension being a suitable tension for using the baseline circumference indicator for determining the baseline circumference of the body part.

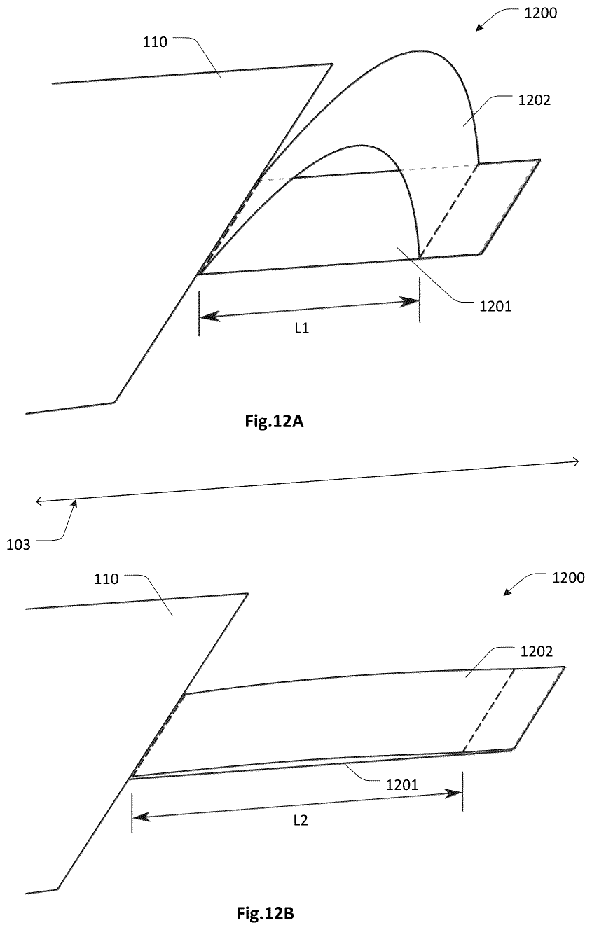

Various configurations of baseline tension indicators are illustrated in FIGS. 12A to 17, which may be incorporated into or used with any of the embodiments of compression band or garments described. Like features are indicated with like reference numerals.

Referring to FIGS. 12A-12B, the baseline tension indicator 1200 comprises a first sheet 1201 of resiliently stretchable material coupled in parallel to a second sheet 1202 of relatively inextensible material such that the first sheet 1201 (length L1) is shorter than the second sheet 1202 (length L2) in the circumferential direction 103 of the band 110, when not under tension.

The first and second sheets 1201, 1202 are arranged in series with the rest of the band 110 and configured such that, when the baseline tension is applied to the baseline tension indicator 1200 and the band 110 for determining the baseline circumference using the baseline circumference indicator, the first sheet 1201 is extended to the same circumferential length (L2) as the second sheet 1202. That is, the first and second sheets 1201, 1202 are pulled taught to come together.

The first sheet 1201 may be formed of an elastic fabric having a higher elasticity than the elastic portion of the band, so that use of the baseline tension indicator does not significantly strain the elastic portion of the band. The width and elasticity of the first sheet 1201 may be chosen to correspond to a selected baseline tension.

The second sheet 1202 may be formed of a relatively inextensible material, for example, the same material as the relatively inextensible portion of the band.

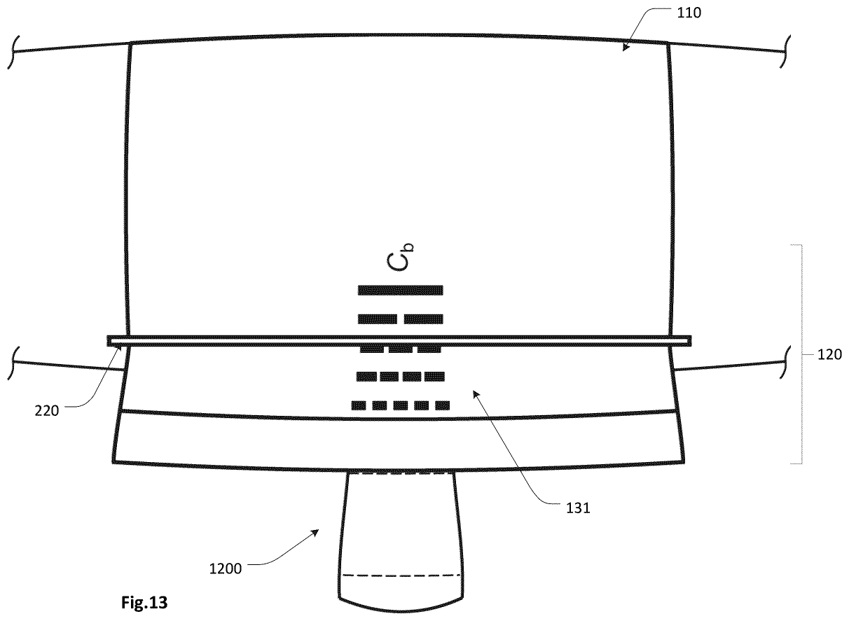

FIG. 13 illustrates the baseline tension indicator 1200 incorporated into the compression band 200 in a similar configuration to that shown in FIG. 2C. The baseline tension indicator 1200 is coupled to the free end of the band for use in tensioning the band to the baseline tension during determination of the baseline circumference.

The width of the baseline tension indicator 1200 may be less than the width of the band 110 and take up tension in line with the corresponding portion of the band 110.

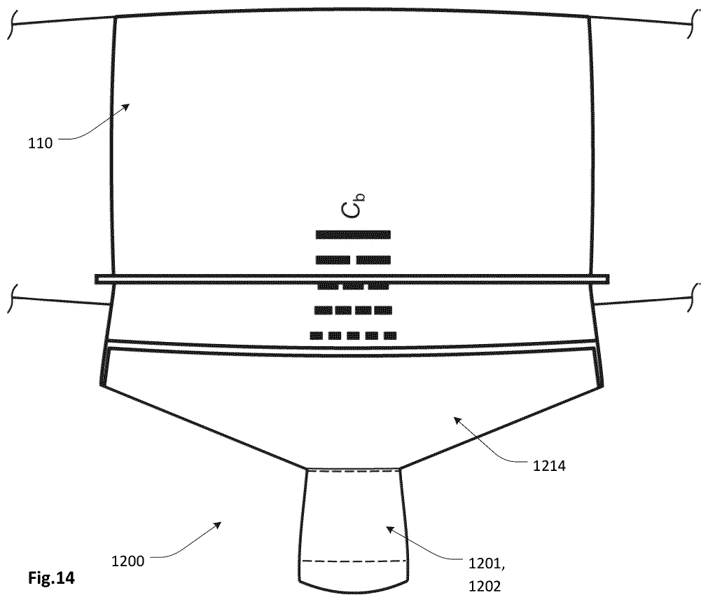

An alternative configuration for incorporating the baseline tension indicator 1200 is shown in FIG. 14 in a similar configuration to that shown in FIG. 2C. In this embodiment, the baseline tension indicator 1200 comprises a spreader 1214 which is relatively rigid and configured to distribute the tension from the full width of the band to the relatively more narrow sheets 1201, 1202 of the baseline tension indicator 1200.

In some embodiments, the baseline tension indicator may be removably coupled to part of the band, such as the free end of the band, as shown in FIG. 14. For example, the baseline tension indicator may be provided as part of a kit or assembly along with one or a plurality of compression bands or a garment for use with the plurality of compression bands or different compression bands in a garment.

The baseline tension indicator may be removably coupled to the band via any of the fastening means described, including, hook-and-loop fasteners such as Velcro, larger hooks and eyelets, or zippers, for example.

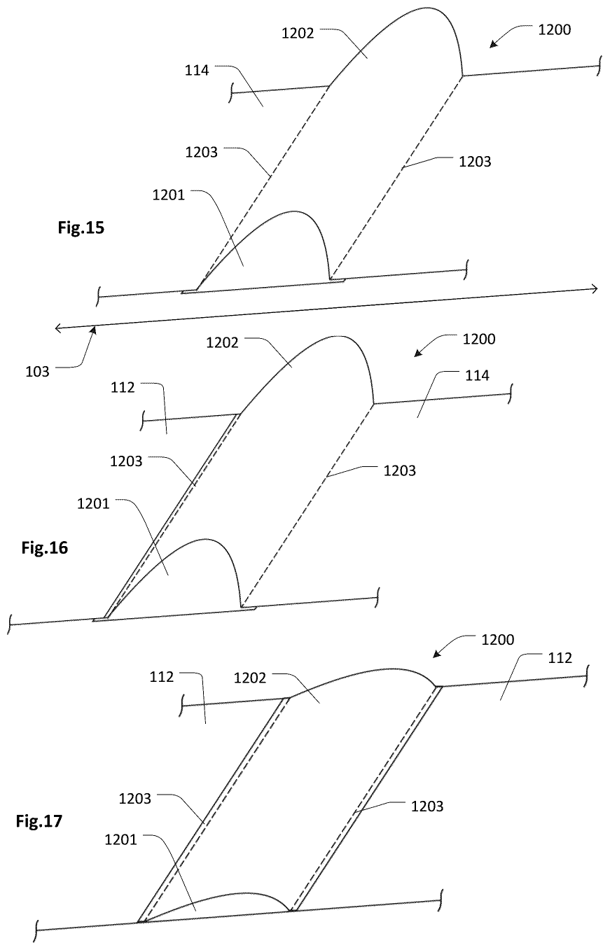

In some embodiments, the baseline tension indicator may be incorporated into the band of the compression band and extend across the full width of the band. This may be beneficial for embodiments where there is no free end, or no easily accessible free end of the band. For example, continuous bands and/or laced-up bands. FIGS. 15 to 17 illustrate some examples of full width baseline tension indicators, according to some embodiments. These and other configurations of baseline tension indicators may be incorporated into any of the described embodiments of compression bands and/or garments. In these embodiments, the baseline tension is indicated when the baseline tension indicator is smoothed out against the body part in line with the rest of the band.

FIG. 15 illustrates a baseline tension indicator 1200 incorporated into the inextensible part of a band. The second sheet 1202 is continuous with the inextensible part of the band 114 and the first sheet 1201 is stitched to the inextensible part of the band 114 (and second sheet 1202) at the parallel stitching lines 1203.

FIG. 16 illustrates a baseline tension indicator 1200 incorporated into the inextensible part of a band at the boundary with the resiliently stretchable part of the band 112. The second sheet 1202 is continuous with the relatively inextensible part of the band 114 and the first sheet 1201 is stitched to the inextensible part of the band 114 (and second sheet 1202) at the parallel stitching lines 1203.

FIG. 17 illustrates a baseline tension indicator 1200 incorporated into the resiliently stretchable part of a band 112. The first sheet 1201 is continuous with the resiliently stretchable part of the band 112 and the second sheet 1202 is stitched to the resiliently stretchable part of the band 112 (and first sheet 1201) at the parallel stitching lines 1203. There is less difference between the circumferential lengths of the first and second sheets 1201, 1202 compared with the examples shown in FIGS. 15 and 16, as the resiliently stretchable part of the band 112 allows relatively less strain.

The illustrations of the baseline tension indicator 1200 are not necessarily to scale, and the relative difference in circumferential length of the first and second sheets 1201, 1202 may be selected to suit a particular application. Even a small difference may be sufficient if it is clearly evident when the baseline tension is applied to the band. That is, when there is a clear gap between the first and second sheets 1201, 1202 when not under tension, and the gap is closed at the baseline tension.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An adjustable compression band comprising:
a band of flexible material configured to extend circumferentially around a body part of a user, at least part of the band being resiliently stretchable in the circumferential direction;
a baseline circumference indicator for determining a baseline circumference of the body part; and
an adjustment mechanism for adjusting a tension of the band in the circumferential direction to apply pressure to the body part,
wherein the adjustment mechanism comprises an inextensible element, which is relatively inextensible in comparison with the resiliently stretchable part of the band, and which comprises a tension indicator for indicating a target tension adjustment setting corresponding to the determined baseline circumference, the target tension adjustment setting corresponding to a tension required for the band to apply a predetermined interface pressure to the body part based on the determined baseline circumference.

2. The compression band of claim 1, wherein the inextensible element comprises the baseline circumference indicator.

3. The compression band of claim 1, wherein the adjustment mechanism comprises a ratcheting tensioner and the inextensible element comprises a tensioning dial of the tensioner with the tension indicator comprising markings on the dial.

4. The compression band of claim 1, wherein the inextensible element comprises a relatively inextensible part of the band with the tension indicator comprising markings on the relatively inextensible part of the band.

5. The compression band of claim 1, wherein the inextensible element comprises a relatively inextensible cord coupling ends of the band together with the tension indicator comprising markings on the inextensible cord.

6. The compression band of claim 1, further comprising a baseline tension indicator configured to indicate when a predetermined threshold baseline tension is applied to the band, the baseline tension being a suitable tension for using the baseline circumference indicator for determining the baseline circumference of the body part.

7. The compression band of claim 6, wherein the baseline tension indicator comprises a first sheet of resiliently stretchable material coupled in parallel to a second sheet of relatively inextensible material such that the first sheet is shorter than the second sheet in the circumferential direction of the band, when not under tension, and
wherein the first and second sheets are arranged in series with the rest of the band and configured such that, when the baseline tension is applied to the baseline tension indicator and the band for determining the baseline circumference using the baseline circumference indicator, the first sheet is extended to the same circumferential length as the second sheet.

8. The compression band of claim 1, wherein a length of the resiliently stretchable part of the band in the circumferential direction varies across a lateral width of the band.

9. The compression band of claim 8, wherein a circumference of the band varies across a lateral width of the band.

10. The compression band of claim 9, wherein the varying circumference of the band across the lateral width of the band is configured such that the predetermined interface pressure applied to the body part varies across the width of the band.

11. The compression band of claim 1, wherein the predetermined interface pressure associated with the target tension is a first predetermined pressure associated with a first target tension and corresponding target tension adjustment settings associated with corresponding baseline circumferences, and
wherein the tension indicator further comprises a second target tension with corresponding target tension adjustment settings associated with corresponding baseline circumferences for the band to apply a second predetermined interface pressure to the body part.

12. The compression band of claim 11, wherein the baseline circumference indicator comprises a set of baseline circumference markings, and the tension indicator comprises a set of target tension markings, each of the target tension markings indicating the target tension adjustment setting associated with the baseline circumference of a corresponding one of the baseline circumference markings to apply the corresponding predetermined interface pressure.

13. The compression band of claim 12, wherein the target tension markings comprise a first set of markings corresponding to the first predetermined pressure, and a second set of markings corresponding to the second predetermined pressure.

14. The compression band of claim 12, wherein each set of markings includes a plurality of markings covering a range of baseline circumferences to allow for expected dimensional changes of the body part.

15. The compression band of claim 12, wherein each of the markings in each set of markings includes a visual similarity with the or each corresponding marking in the or each other set of markings.

16. (canceled)

17. The compression band of claim 1, wherein the adjustment mechanism is configured to adjust the distance in the circumferential direction between two parts of the band, and
wherein the adjustment mechanism comprises fasteners disposed at a range of circumferential locations on the band allowing one part of the band to be fastened to another part of the band to select and set the tension of the band.

18.-22. (canceled)

23. A garment comprising one or more ones of the compression band of claim 1.

24. The garment of claim 23, further comprising non-adjustable portions connected to the one or more compression bands.

25. The garment of claim 23 or 24, comprising a plurality of ones of the compression band arranged in parallel and integrally formed with each other.

26. A method of applying an interface pressure to a body part of a user, the method comprising:
placing the compression band of claim 1 around the body part of the user;
adjusting the circumference of the band to a neutral configuration to determine a baseline circumference of the body part indicated by the baseline circumference indicator; and
adjusting the tension of the band to a corresponding target tension indicated by the target tension indicator to apply the predetermined pressure to the body part.

27. (canceled)
